(19) United States
(12) Patent Application Publication
(10) Pub. No.: US 2024/0358751 A1
(43) Pub. Date: Oct. 31, 2024

(54) IPSC-DERIVED IMMUNE CELLS IN PROPHYLAXIS AND TREATMENT OF AGE-ASSOCIATED AND NEURODEGENERATIVE DISEASES

(71) Applicant: Cedars-Sinai Medical Center, Los Angeles, CA (US)

(72) Inventors: Vanessa Alexandra Moser, Los Angeles, CA (US); Clive Niels Svendsen, Pacific Palisades, CA (US); Helen Goodridge, Montrose, CA (US); Alexander LaPerle, North Hollywood, CA (US)

(73) Assignee: Cedars-Sinai Medical Center, Los Angeles, CA (US)

(21) Appl. No.: 18/681,641

(22) PCT Filed: Aug. 19, 2022
(86) PCT No.: PCT/US2022/040921

§ 371 Date: Feb. 6, 2024

Related U.S. Application Data
(60) Provisional application No. 63/234,984, filed on Aug. 19, 2021.

(51) Int. Cl. A61K 35/15; A61P 25/28; C12N 5/0786

(52) U.S. Cl. CPC A61K 35/15; A61P 25/28; C12N 5/0645; C12N 2501/115; C12N 2501/125; C12N 2501/145; C12N 2501/15; C12N 2501/155; C12N 2501/165; C12N 2501/22; C12N 2501/2303; C12N 2501/2334; C12N 2501/26; C12N 2501/845; C12N 2506/45

(57) ABSTRACT

Mononuclear phagocytes derived from induced pluripotent stem cells, denoted as iMPs, which comprise monocytes generated from the induced pluripotent stem cells and optionally further macrophages generated from the induced pluripotent stem cells, are provided for use in improving cognitive function, improving neural health, and/or alleviating or treating a neurodegenerative disorder in a mammal In various embodiments, the iMPs produce macrophages after transplantation or after being stimulated in vitro, and/or express macrophage markers. We showed that iMPs upon administration improve cognition and neural healthy in rodent models of aging, Alzheimer's disease, and amyotrophic lateral sclerosis. Treatment methods are also provided using mononuclear phagocytes generated from autologous stem cells or from induced pluripotent stem cells obtained from autologous cells in patients in need of treatment or prophylaxis of a neurodegenerative disorder.

### CROSS-REFERENCE TO RELATED APPLICATIONS

This application includes a claim of priority under 35 U.S.C. § 119 (e) to U.S. provisional patent application No. 63/234,984, filed Aug. 19, 2021, the entirety of which is hereby incorporated by reference.

### FIELD OF INVENTION

This invention relates to pluripotent stem cell-derived therapies for neurodegenerative diseases and aging, and an improved protocol for producing pluripotent stem cell-derived monocytes and/or macrophages.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

A neurodegenerative disease affects nerve cells in the brain or the peripheral nervous system, which will lose function over time. As an example, Alzheimer disease (AD) is a progressive neurodegenerative disease that affects cognition and function, where patients experience symptoms affecting multiple aspects of life, such as cognitive function, behavior, mood, and psychological condition. However, there is no known disease-modifying therapy, making drug discovery an area of unmet medical. As another example, amyotrophic lateral sclerosis (ALS), also known as Lou Gehrig disease, is a common, devastating, and invariably fatal adult neurodegenerative disease. In addition to the loss of upper and lower motor neurons, ALS is now regarded as a disorder with immune dysregulation, which is characterized by alterations/activation of inflammatory cells that augment disease burdens and rates of disease progression. Unfortunately, no treatments are presently available to arrest or substantially delay these inexorable inflammatory responses in patients with ALS.

Previous studies using young plasma and bone marrow have demonstrated some improvement in the cognitive performance and neural health in aging adults or those with neurodegeneration. However, risks associated with administering young plasma and bone marrow make them unsuitable therapeutics. For instances, plasma infusion can carry the risks such as allergies and transfusion-related circulatory overload, resulting in pulmonary edema (swelling) and difficulty in breathing.

Therefore, it is an objective of the present invention to provide new therapies for treatment or alleviation of neurodegenerative disorders.

### SUMMARY OF THE INVENTION

The following embodiments and aspects thereof are described and illustrated in conjunction with compositions and methods which are meant to be exemplary and illustrative, not limiting in scope.

In various embodiments, methods for treating or providing prophylaxis for a subject are provided, including administering a therapeutically effective quantity of mononuclear phagocytes generated from pluripotent stem cells to the subject, wherein the subject has a neurodegenerative disorder, experiences cognitive impairment, or is in need of cognitive function improvement. Mononuclear phagocytes may include monocytes, macrophages, or a mixture of monocytes and macrophages. A therapeutically effective quantity of mononuclear phagocytes for a human subject may be in the order of 1×10⁶, 1×10⁷, or 1×10⁸, given in one or more doses. Therefore, mononuclear phagocytes generated from pluripotent stem cells, especially generated from induced pluripotent stem cells (iPSCs), of the invention herein provide for a large supply of quantities, more superior to naturally occurring counterparts as the latter are difficult, if not impossible, to proliferate in vitro.

In various embodiments, the mononuclear phagocytes for use in treatment disclosed herein are generated from pluripotent stem cells, preferably from iPSCs, in a process comprising culturing the pluripotent stem cells in a cell culture medium under conditions that induce myeloid differentiation, leading to the generation of mononuclear phagocytes. In various embodiments, the process of inducing myeloid differentiation so as to generate the mononuclear phagocytes does not include driving the cells into microglial or dendritic cells. In some additional embodiments, the process of inducing myeloid differentiation so as to generate the mononuclear phagocytes does not include driving the cells into macrophages in vitro; whereas in other additional embodiments, the process of inducing myeloid differentiation so as to generate the mononuclear phagocytes includes driving the cells into macrophages in vitro.

In some embodiments, the process of inducing myeloid differentiation so as to generate the mononuclear phagocytes includes the first one, two, three, or all four steps of: contacting the iPSCs with a first composition comprising bone morphogenetic protein (BMP-4) in a medium; contacting the cell culture medium with a second composition comprising one or more of bFGF, VEGF, and SCF, after culturing of the iPSCs in the presence of the first composition; contacting the cell culture medium with a third composition comprising one or more of SCF, IL-3, thrombopoietin (TPO), macrophage colony-stimulating factor (M-CSF), and Fms-like tyrosine kinase 3 ligand (FLT3 ligand), after culturing of the iPSCs in the presence of the second composition; and contacting the cell culture medium with a fourth composition comprising one or more M-CSF, GM-CSF, and FLT3 ligand, after culturing of the iPSCs in the presence of the third composition.

In preferable embodiments, the first composition comprising the BMP-4 is in a medium with bFGF and TGFβ, and optionally further with aminobutyric acid (GABA), pipecolic acid, and lithium chloride. Preferably the first composition is in a mTeSR1 medium. In additional embodiments, the second composition, the third composition, and/or the fourth composition are in a hematopoietic cell medium, such as StemPro-34 medium. Preferably, the medium is serum-free medium, for example serum-free mTeSR1 medium or StemPro-34 serum-free medium.

Additional embodiments provide that the mononuclear phagocytes for use in treatment disclosed herein are generated from iPSCs reprogrammed from blood cells, such as peripheral blood mononuclear cells, or from fibroblast or another somatic cell source. In some embodiments, the mononuclear phagocytes for use in treatment disclosed herein are autologous, i.e., generated from iPSCs reprogrammed from autologous somatic cells. In some embodiments, the mononuclear phagocytes for use in treatment of a subject disclosed herein are generated from iPSCs reprogrammed from autologous somatic cells obtained from the subject.

In various embodiments, the generated mononuclear phagocytes are for use in an aging mammalian subject, or in a subject with a neurodegenerative disorder such as Alzheimer's disease, amyotrophic lateral sclerosis (ALS), Parkinson's disease, multiple sclerosis (MS), schizophrenia, and autism spectrum disorders, or in a subject in need of reducing inflammation related to the neurodegenerative disorder. In various embodiments, the mononuclear phagocytes generated as disclosed herein provides for improved cognitive functions in the subject in one or more behavior assessment, in levels of synaptic transporter, VGLUT1, in microglial branching length, or another molecular analysis. In some aspects, the improvement is compared to the baseline condition of the subject prior to receiving the administration of the mononuclear phagocytes. In some aspects, the improvement results in a comparable or similar level to a healthy subject who is young, or free from a neurodegenerative disorder.

Mononuclear phagocytes generated from pluripotent stem cells are also provided, which may be in a composition that further includes one or more pharmaceutically acceptable excipients. Preferably, mononuclear phagocytes generated from iPSCs reprogrammed from blood cells or fibroblasts are provided.

Additional embodiments provide methods for drug screening using the mononuclear phagocytes generated herein, including but not limited to high-throughput screening methods. In some embodiments, a method is provided for identifying a compound useful in the treatment or prevention of a disease or disorder associated with a defect in or deficiency of monocytes and/or macrophages, or a neurodegenerative disease or disorder, wherein the method includes contacting a mononuclear phagocyte generated by a method disclosed herein with a candidate compound, and determining whether the candidate compound improves the defect in or deficiency of monocytes or macrophages, or the neurodegenerative disease or disorder, respectively.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a schematic of a mouse model, where we administer iPSC-derived mononuclear phagocytes every third day and perform behavioral testing on a number of cognitive tasks. “83iGFP” stands for the iPSC line used to generate iMPs. “SAB” stands for spontaneous alternation behavior test. “FC” stands for fear conditioning. “NOP” stands for novel object placement test. “NOR” stands for novel object recognition test. “EPM” stands for elevated plus maze. Young mice are 3-4 months of age while aged mice are 11-13 months old.

FIG. 2 is a schematic depicting a spontaneous alternation behavior (SAB) task, which tests the spatial working memory in which a mouse with good spatial working memory will rotate from arm A to B to C, while those with poorer memory will alternate more frequently between arms they just came out of (for example, going between arm A and B and back to A).

FIG. 3A shows that aged animals treated with vehicle without iMPs (denoted “Aged Veh”; dots in green) performed worse at the spontaneous alternation behavior task, whereas aged mice treated with iMPs (denoted “Aged iMPs”; dots in red) did not perform worse, compared to young animals (denoted “Young”; dots in blue). FIG. 3B shows that both aged groups made significantly less arm entries than young animals, indicating that the effect of the iMPs in FIG. 3A is cognitive, and not due to changes in locomotion.

FIG. 4A depicts a “novel object recognition” (NOR) study, in which mice are exposed to two novel objects they have never encountered before, and after a 30 min retention delay, they are then exposed to one new object—if they remember seeing the previous object before they will spend more time with the novel one. There were no age or treatment effects in the novel object recognition assay—the results are not statistically different among the young mice group, the aged mice group treated with vehicle, and the aged mice group treated with the iMPs.

FIG. 4B depicts a “novel object location/placement” (NOP) study, in which one of the two objects is moved and the animal will spend more time at the one located in a novel location if it remembers where the previous objects were placed. Results show that aged mice were significantly impaired at recognizing the object that had moved locations, and that iMP treatment (“Aged iMPs” group) significantly improved the performance of aged mice in this task.

FIG. 5A depicts that the number of Neun+ cells does not change with either age or treatment in Cornu Ammonis areas 1 and 3 (CA1, CA3). Neun is a marker of neuronal nuclei, and thus indicative of neuron number.

FIG. 5B depicts that, relative to young animals, VGLUT1 is decreased in aged animals but not in those treated with iMPs. VGLUT1 is a glutamate transporter located at synapses, is essential for normal synaptic function, and has been shown to decrease in Alzheimer's disease.

FIG. 6A depicts that in CA3, microglia branch length is reduced in aging animals but not in aging animals treated with iMPs. Aging animals treated with iMPs are denoted as “Aged iMPs” group. FIG. 6B depicts that in CA1, branch length is again decreased in aging animals but is significantly increased in aging animals treated with iMPs. Microglia are the main immune cell of the brain and given that their function is to survey the environment for damage or insults, they usually have long, branching processes. However, when activated, they retract these processes and will have less branch length per cell. This is known to happen both with aging and Alzheimer's disease. Additionally, in both cases there is an increase in overall microglia number.

FIG. 7 depicts that LAMP1 is increased in CA1 in both aging groups. LAMP1 is a lysosomal marker that has been shown to increase with aging and Alzheimer's disease.

FIG. 8 depicts that the number of astrocytes is increased in aging animals, but not in aging animals treated with iMPs. GFAP is a marker of astrocytes, which normally increase in number and in cell body size with aging and in disease.

FIG. 9A depicts a timeline of a study of iMPs administered in a mouse model of Alzheimer's disease, starting with 5xFAD mice at 3 months of age, which is when they first develop pathology. (AD mouse model with amyloid & microglial activation starts around 2 months.) Cyclosporine A is administered via intraperitoneal injection 3 days prior to first cells, then via drinking water. 83iGFP mononuclear cytes are injected at 500,000 cells/injection for eight injections as indicated in the figure. We will be repeating this in 7 month-old animals that have extensive pathology already.

FIG. 9B depicts that 5xFAD mice treated at 3 months with iMPs (“iMPs”) show no changes on tasks of spatial working memory and of short term spatial memory, compared with 5xFAD mice treated with vehicle. FIG. 9C depicts that iMP-treated animals show an improvement in the “novel object recognition” study.

FIG. 10A is a hierarchical clustering of bulk RNA-Sequencing data comparing iPSCs (denoted as number 1, with triplicates denoted as 1A, 1B, and 1C), iMPs grown in a well plate (as described in Example 2; denoted as number 3, with triplicates denoted as 3A, 3B, 3C), cryopreserved iMPs collected from cultures from the well plate (denoted as number 2, with triplicates denoted as 2A, 2B, 2C), and iMPs produced in a bioreactor at early time (day 15; denoted as number 4, with triplicates denoted as 4A, 4B, 4C) and at late time (day 55; denoted as number 5, with triplicates denoted as 5A, 5B, 5C). All of the differentiated iMPs (groups 2-5) are very similar to each other.

FIG. 10B is a principal component analysis plot depicting the 2000 most variable genes across the assay depicted in FIG. 10A. Like the clustering in FIG. 10A, proximity indicates similarity, and therefore the iMPs in groups 2-5 were shown to be similar to each other.

FIG. 11 shows RNA-seq results of the expression (in transcripts per kilobase million, TPM) of some key monocyte/macrophage markers (CD14, CD16, CD64, CD11b, CD11c, and CD71) in groups 2-5 of the cells depicted in FIG. 10A.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

A “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In an embodiment, the subject is human. In a further embodiment, the subject is a human exhibiting signs of neurodegenerative symptoms or diseases, e.g., showing signs of loss of memory (short-term memory, working memory, etc.), signs of confusion with time or place, signs of tremor.

“Neurological disorders” refer to disorders that affect the brain as well as the nerves found throughout the body and the spinal cord, which include but are not limited to epilepsy, learning disabilities, neuromuscular disorders, autism, attention deficit disorder, brain tumors, and cerebral palsy.

“Neurodegenerative diseases or disorders” generally describe a pathology where nerve cells in the brain or peripheral nervous system lose function over time and ultimately die. The risk of being affected by a neurodegenerative disease increases dramatically with age. Alzheimer's disease and Parkinson's disease are common neurodegenerative diseases. Examples of neurodegenerative diseases include Alzheimer's disease and other dementias, Parkinson's disease and its related disorder, Huntington's disease, Prion disease, motor neuron disease, spinocerebellar ataxia, spinal muscular atrophy, and amyotrophic lateral sclerosis.

“Spatial working memory” entails the ability to keep spatial information active in working memory over a short period of time.

“Short-term memory,” also known as primary or active memory, is the capacity to store a small amount of information in the mind and keep it readily available for a short period of time. Usually short-term memory is very brief. When short-term memories are not rehearsed or actively maintained, they can last mere seconds.

The terms “treating” or “treatment” or “to treat” refer to therapeutic measures that cure, slow down, lessen symptoms of, and/or halt progression of a diagnosed pathologic disease or disorder. Thus, those in need of treatment include those already with the disorder. In certain embodiments, a subject is successfully “treated” for a disease or disorder if the subject shows, e.g., total, partial, permanent, or transient, alleviation or elimination of any symptom associated with the disease or disorder.

The term “about” or “approximately” when used in connection with a referenced numeric indication (in percentage) means the referenced numeric indication (in percentage) plus or minus up to 5% of that referenced numeric indication (in percentage), unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims. In other embodiments, “about” or “approximately” when used in connection with a referenced numeric indication of a period of time in units of at least days (e.g., day, week, or month) means the referenced numeric indication plus or minus at least one day, or at least one day and up to 10% of the indicated period of time when 10% of the indicated period of time is greater than 1 day. For example, the language “approximately 4 days covers the range of 3 days to 5 days; the language “approximately” 60 days or “approximately” 2 months covers the range of 54 days to 66 days.

The term “pluripotent stem cells” or “PSCs” refer to self-replicating cells that have the ability to develop into any of endoderm, ectoderm, and mesoderm cells, as well as growth ability. Examples of the pluripotent stem cells include, but are not limited to, embryonic stem (ES) cells, embryonic stem cells derived from a cloned embryo obtained by nuclear transfer (ntES cells), germline stem cells (“GS cells”), embryonic germ cells (“EG cells”), and induced pluripotent stem cells (“iPS cells” or “iPSCs”). In some embodiments PSCs are human PSCs. Preferred examples of the PSCs include ES cells and iPS cells.

ES cells are stem cells established from the inner cell mass of an early embryo (for example, blastocyst) of a mammal such as human or mouse, and ES cells have pluripotency and growth ability by self-renewal. ES cells can be established by removing the inner cell mass from the blastocyst of a fertilized egg of the subject animal, followed by culturing the inner cell mass on fibroblasts as feeders. The cells can be maintained by subculturing using a medium supplemented with substances such as leukemia inhibitory factor (LIF) and/or basic fibroblast growth factor (bFGF). Methods of establishment and maintenance of human and monkey ES cells are described in, for example, U.S. Pat. No. 5,843,780 B; Thomson J A, et al. (1995), Proc Natl. Acad. Sci. U S A. 92:7844-7848; Thomson J A, et al. (1998), Science. 282:1 145-1147; H. Suemori et al. (2006), Biochem. Biophys. Res. Commun., 345:926-932; M. Ueno et al. (2006), Proc. Natl. Acad. Sci. USA, 103:9554-9559; H. Suemori et al. (2001), Dev. Dyn., 222:273-279; H. Kawasaki et al. (2002), Proc. Natl. Acad. Sci. USA, 99:1580-1585; and Klimanskaya I, et al. (2006), Nature. 444:481-485.

Induced pluripotent stem (iPS) cells can be prepared by introducing specific reprogramming factors to somatic cells, which reprogramming factors are in the forms of DNAs or proteins. iPS cells are somatic cell-derived artificial stem cells having properties almost equivalent to those of ES cells, such as pluripotency of differentiation and growth ability by self-renewal. The reprogramming factors may be constituted by genes or gene products thereof, or non-coding RNAs, which are expressed specifically in ES cells; or genes or gene products thereof, non-coding RNAs or low molecular weight compounds, which play important roles in maintenance of the undifferentiated state of ES cells. Examples of the genes of the reprogramming factors include Oct3/4, Sox2, Soxl, Sox3, Soxl5, Soxl7, Klf4, Klf2, c-Myc, N-Myc, L-Myc, Nanog, Lin28, Fbxl5, ERas, ECAT15-2, Tell, beta-catenin, Lin28b, Salll, Sall4, Esrrb, Nr5a2 and Tbx3, and these reprogramming factors may be used either alone or in combination.

The term “serum” refers to human serum, monkey serum, fetal bovine serum, bovine serum, pig serum, equine serum, donkey serum, chicken serum, quail serum, sheep serum, goat serum, dog serum, cat serum, rabbit serum, rat serum, guinea pig serum, mouse serum, and the like. Examples of the medium which does not contain serum include minimum essential medium (MEM), Dulbecco's modified Eagle's medium (DMEM), Iscove's modification of Dulbecco's medium (IMDM), StemPro-34SFM (Invitrogen), Stemline II (Sigma-Aldrich) and the like which are supplemented with ITS; medium for culturing primate ES cells (medium for primate ES/iPS cells, ReproCELL) wherein a serum alternative has been preliminarily added; and serum-free medium (mTeSR, Stemcell Technology). The medium which does not comprise serum, or “serum-free”, is more preferably mTeSR1 medium or StemPro-34 serum-free medium.

“Hematopoietic factor” refers to a factor that promotes differentiation and growth of blood cells. Examples thereof include the stem cell factor (SCF), granulocyte-colony stimulating factor (G-CSF), granulocyte-monocyte colony-stimulating factor (GM-CSF), macrophage colony-stimulating factor (M-CSF), erythropoietin (EPO), thrombopoietin (TPO), interleukins, and Flt3 ligand. Interleukins are proteins secreted from leukocytes, and can be divided into various types such as IL-1, IL-2, IL-3, IL-4, IL-5, IL-6, IL-7, IL-8 and IL-9.

The phrase “substantially pure” refers to a population of cells wherein at least 95% of the cells have the recited phenotype or expression marker profiles. In all embodiments that refer to a “substantially pure” cell population, alternative embodiments in which the cell populations have a lower or higher level of purity are also contemplated. For example, in some embodiments, instead of a given cell population being “substantially pure” the cell population may be one in which at least 40%, 45%, 50%, 55%, 60%, 65%, 70%, 75%, 80%, 85%, 90%, 95%, 96%, 97%, 98%, or 99% of the cells, or 100% of the cells, have the recited phenotype or gene expression profiles.

Various embodiments provide methods for improving cognitive function in a subject, or treating a subject with a neurodegenerative disorder, or alleviating, treating, or delaying onset of a neurodegenerative disorder, or reducing inflammation in a subject with a neurodegenerative disorder in a subject, wherein the methods include administering to the subject a therapeutically effective amount of a composition comprising a population of mononuclear phagocytes generated from pluripotent stem cells. In some aspects, the population of mononuclear phagocytes are differentiated from induced pluripotent stem cells (iPSCs). In other aspects, the population of mononuclear phagocytes are differentiated from autologous iPSCs. In yet another aspect, the population of mononuclear phagocytes are differentiated from an embryonic stem cell. In various embodiments, the mononuclear phagocytes generated from pluripotent stem cells comprise monocytes generated from the pluripotent stem cells. In various embodiments, the mononuclear phagocytes generated from pluripotent stem cells are monocytes generated from the pluripotent stem cells. In some embodiments, the mononuclear phagocytes generated from pluripotent stem cells further comprise macrophages; and the macrophages are produced after transplanting the monocytes generated from the pluripotent stem cells or by stimulation in vitro or ex vivo of the monocytes generated from the pluripotent stem cells. In further embodiments, the population of mononuclear phagocytes are myeloid-lineage cells generated from iPSCs by one or more differentiation methods disclosed herein. In additional embodiments, the mononuclear phagocytes of the invention herein include monocytes, may further include macrophages, but excludes neutrophils.

In some embodiments, methods are provided for improving cognitive function in a subject, or treating a subject with a neurodegenerative disorder, or alleviating, treating, or delaying onset of a neurodegenerative disorder, or reducing inflammation in a subject with a neurodegenerative disorder in a subject, wherein the methods include administering to the subject a therapeutically effective amount of a composition comprising monocytes generated from iPSCs by one or more differentiation methods disclosed herein. In some embodiments, the methods for improving cognitive function in a subject, or treating a subject with a neurodegenerative disorder, or alleviating, treating, or delaying onset of a neurodegenerative disorder, or reducing inflammation in a subject with a neurodegenerative disorder in a subject, include administering to the subject a therapeutically effective amount of a composition comprising cells consisting of monocytes generated from iPSCs by a differentiation method disclosed herein, wherein the differentiation method does not include differentiating the generated monocytes to macrophages in vitro, e.g., the differentiation method does not include culturing the generated monocytes in the presence of M-CSF and one or both of IFN-gamma or IL-4. In other embodiments, the methods for improving cognitive function in a subject, or treating a subject with a neurodegenerative disorder, or alleviating, treating, or delaying onset of a neurodegenerative disorder, or reducing inflammation in a subject with a neurodegenerative disorder in a subject, include administering to the subject a therapeutically effective amount of a composition comprising mononuclear phagocytes generated from iPSCs, which may be (1) a mixture of monocytes generated from the iPSCs and macrophages generated from the iPSCs, if the differentiation method further drives macrophage differentiation in vitro, or (2) substantially purely macrophages generated from the iPSCs, if the differentiation method further drives macrophage differentiation in vitro and additional sorting/purification is performed to obtain only macrophages generated from the iPSCs, or (3) substantially purely monocytes generated from the iPSCs, if the differentiation method does not drive differentiation of the monocytes generated from the iPSCs.

In various embodiments, a clinically meaningful amount of mononuclear phagocytes (or myeloid monocytic cells) are generated from the pluripotent stem cells (e.g., induced pluripotent stem cells) in a process disclosed herein, especially via culturing in a bioreactor. A clinically meaningful amount of mononuclear phagocyte generated from the pluripotent stem cells, especially from iPSCs, can be stored (e.g., frozen) or maintained in culturing, for use in patient administration in a significant amount, as opposed to having to isolate mononuclear phagocytes (or monocytes) from the patient and use them. Starting from pluripotent stem cells, specifically induced pluripotent stem cells, the generated mononuclear phagocyte described herein may be less prone to genetic mutations, and may have fewer disease mutations, compared to autologous monocytes or macrophages obtained from a patient or subject in need of the treatment.

In some embodiments, a method for improving cognitive function in a subject, especially an aging subject (e.g., human of an age between 40 and 50, between 50 and 60, between 60 and 70, between 70 and 80, between 80 and 90, between 90 and 100, or greater than 100 years old), includes administering to the subject a therapeutically effective amount of a composition comprising mononuclear phagocytes generated from the subject's autologous iPSCs. In some embodiments, a method for treating a subject with a neurodegenerative disorder includes administering to the subject a therapeutically effective amount of a composition comprising mononuclear phagocytes differentiated from the subject's autologous iPSCs. In some embodiments, a method for alleviating, treating, or delaying onset of a neurodegenerative disorder in a subject includes administering to the subject a therapeutically effective amount of a composition comprising mononuclear phagocytes differentiated from the subject's autologous iPSCs. In further embodiments, a method for treatment or prevention includes administering mononuclear phagocytes generated from autologous iPSCs to a subject having, suspected of having, or at risk of developing a disease or disorder associated with a defect in or deficiency of mononuclear phagocytes. In some embodiments, a method for treatment or prevention includes administering mononuclear phagocytes generated from autologous iPSCs to a subject having, suspected of having, or at risk of developing a disease or disorder associated with a defect in or deficiency of macrophages, wherein the administered mononuclear phagocytes produce macrophages after administration into the subject. In yet additional embodiments, a method for reducing inflammation includes administering mononuclear phagocytes generated from autologous iPSCs to a subject having, suspected of having, or at risk of developing a disease or disorder associated with inflammation; optionally wherein the administered mononuclear phagocytes produce macrophages after the administration in the subject. A disease or disorder associated with inflammation may be a neurodegenerative disease or disorder.

In other embodiments, a method for improving cognitive function in a subject, especially an aging subject (e.g., human of an age between 40 and 50, between 50 and 60, between 60 and 70, between 70 and 80, between 80 and 90, between 90 and 100, or greater than 100 years old), includes administering to the subject a therapeutically effective amount of a composition comprising myeloid monocytic cells or monocytes generated from the subject's autologous iPSCs, wherein the myeloid monocytic cells or monocytes are not stimulated to differentiate into microglia, dendritic cells, or macrophages prior to the administration to the subject. In some embodiments, a method for treating a subject with a neurodegenerative disorder includes administering to the subject a therapeutically effective amount of a composition comprising myeloid monocytic cells or monocytes generated from the subject's autologous iPSCs, wherein the myeloid monocytic cells or monocytes are not stimulated to differentiate into microglia, dendritic cells, or macrophages prior to the administration to the subject. In some embodiments, a method for alleviating, treating, or delaying onset of a neurodegenerative disorder in a subject includes administering to the subject a therapeutically effective amount of a composition comprising myeloid monocytic cells or monocytes generated from the subject's autologous iPSCs, wherein the myeloid monocytic cells or monocytes are not stimulated to differentiate into microglia, dendritic cells, or macrophages prior to the administration to the subject. In further embodiments, a method for treatment or prevention includes administering myeloid monocytic cells or monocytes generated from the subject's autologous iPSCs to a subject having, suspected of having, or at risk of developing a disease or disorder associated with a defect in or deficiency of monocytes or mononuclear phagocytes, wherein the myeloid monocytic cells or monocytes are not stimulated to differentiate into microglia, dendritic cells, or macrophages prior to the administration to the subject. In some embodiments, a method for treatment or prevention includes administering myeloid monocytic cells or monocytes generated from the subject's autologous iPSCs to a subject having, suspected of having, or at risk of developing a disease or disorder associated with a defect in or deficiency of macrophages, wherein the administered myeloid monocytic cells or monocytes produces macrophages after administration into the subject. In yet additional embodiments, a method for reducing inflammation includes administering to a subject having, suspected of having, or at risk of developing a disease or disorder associated with inflammation, wherein the myeloid monocytic cells or monocytes are not stimulated to differentiate into microglia, dendritic cells, or macrophages prior to the administration to the subject. A disease or disorder associated with inflammation may be a neurodegenerative disease or disorder. In alternative embodiments, a method for treatment or prevention, or for reducing inflammation, includes administering myeloid monocytic cells from the subject's autologous iPSCs to the subject, wherein the administered myeloid monocytic cells are further differentiated into macrophages prior to the administration into the subject.

In various implementations, the methods including administering the mononuclear phagocytes differentiated from pluripotent stem cells, preferably from iPSCs, do not include administering plasma or bone marrow to the subject; or the subject in these methods do not receive plasma or bone marrow transplant. In alternative implementations, the methods including administering the mononuclear phagocytes differentiated from pluripotent stem cells, preferably from iPSCs, are in addition to a plasma or bone marrow transplant therapy for the subject. In another implementation, the methods including administering the mononuclear phagocytes differentiated from pluripotent stem cells are for a subject whose response to plasma infusions or to bone marrow transplant therapy is ineffective or involves morbidity complications.

In various embodiments of the methods, the mononuclear phagocytes are generated from pluripotent stem cells in a process that comprises culturing the pluripotent stem cells in a cell culture medium under conditions that induce myeloid differentiation, so as to generate myeloid lineage cells (preferably monocytes), and the process does not include contacting or culturing the generated cells in a microglial differentiation medium or a dendritic cell differentiation medium. For example, the process of generating mononuclear phagocytes (or the myeloid lineage cells) from pluripotent stem cells does not include culturing or contacting the generated cells in the presence of (a) IL-34, (b) IL-34 and GM-CSF, (c) IL-4, or (d) IL-4 and GM-CSF. Therefore, the mononuclear phagocytes (preferably monocytes) generated from the pluripotent stem cells for use in one or more methods disclosed herein are not microglia or dendritic cells. Thereby, the methods disclosed herein for improving cognitive function in a subject, or treating a subject with a neurodegenerative disorder, or alleviating, treating, or delaying onset of a neurodegenerative disorder in a subject, or treatment or prevention in a subject having, suspected of having, or at risk of developing a disease or disorder associated with a defect in or deficiency of macrophages or monocytes, do not include administering microglia or dendritic cells generated from the pluripotent stem cells. In additional embodiments, the process further excludes contacting or culturing the generated cells in a macrophage differentiation medium or in the presence of macrophage differentiation stimulants. For example, in these additional instances, the process of generating mononuclear phagocytes (or the myeloid lineage cells) from pluripotent stem cells does not include culturing or contacting the generated cells in the presence of (a) IL-34, (b) IL-34 and GM-CSF, (c) IL-4, (d) IL-4 and GM-CSF, or (e) M-CSF and either one or both of IFN-gamma and IL-4. That is, the process of generating the mononuclear phagocytes does not include culturing the generated cells with any of (a) IL-34, (b) a combination of IL-34 and GM-CSF, (c) IL-4, (d) a combination of IL-4 and GM-CSF, (e) a combination of M-CSF and IFN-gamma, and (f) a combination of M-CSF and IL-4. Multiple reagents in a “combination” may be added to a medium concurrently, or subsequently. Alternatively, the process may further include contacting or culturing the generated cells in a macrophage differentiation medium or in the presence of macrophage differentiation stimulants. For example, the process of generating mononuclear phagocytes (or the myeloid lineage cells) from pluripotent stem cells does not include culturing or contacting the generated cells in the presence of (a) IL-34, (b) IL-34 and GM-CSF, (c) IL-4, or (d) IL-4 and GM-CSF, but does include culturing or contacting the generated cells with M-CSF and either one or both of IFN-gamma and IL-4.

Specifically, in some instances, a process for generating mononuclear phagocytes from pluripotent stem cells includes culturing the pluripotent stem cells in a cell culture medium under conditions that induce myeloid differentiation, wherein the culturing comprises contacting the pluripotent stem cells with a first composition comprising BMP-4 in a serum-free medium with bFGF, with bFGF and TGFβ, or with bFGF, TGFβ, aminobutyric acid, pipecolic acid, and lithium chloride. In various aspects, the culturing comprises contacting the pluripotent stem cells with a first composition comprising BMP-4 in a mTeSR1 medium with all, or one, two, three, or four, of bFGF, TGFβ, aminobutyric acid, pipecolic acid, and lithium chloride.

Culturing the pluripotent stem cells in a cell culture medium under conditions that induce myeloid differentiation may further comprise contacting the cells obtained from the step involving the first composition, with a second composition comprising one or more, or all, factors including bFGF, VEGF, SCF, or a combination thereof. In various aspects, contacting the cells with a second composition refers to changing the cell culture medium to one with the second composition, or contacting the cell culture medium with the second composition. In various aspects, the second composition comprises hematopoietic factors consisting of bFGF, VEGF, or SCF, or a combination of bFGF, VEGF, and SCF; and preferably in a serum-free medium.

Culturing the pluripotent stem cells in a cell culture medium under conditions that induce myeloid differentiation may further comprise contacting the cells obtained from the step involving the second composition, with a third composition comprising one or more, or all, factors including SCF, IL-3, thrombopoietin, M-CSF, FLT3 ligand, or a combination thereof. In various aspects, contacting the cells with a third composition refers to changing the cell culture medium to one with the third composition, or contacting the cell culture medium with the third composition. In various aspects, the third composition comprises hematopoietic factors consisting of SCF, IL-3, thrombopoietin, M-CSF, or FLT3 ligand, or a combination of SCF, IL-3, thrombopoietin, M-CSF, and FLT3 ligand; preferably in a serum-free medium.

Culturing the pluripotent stem cells in a cell culture medium under conditions that induce myeloid differentiation may further comprise contacting the cells obtained from the step involving the third composition, with a fourth composition comprising one or more, or all, factors including M-CSF, GM-CSF, FLT3 ligand, or a combination thereof. In various aspects, contacting the cells with a fourth composition refers to changing the cell culture medium to one with the fourth composition, or contacting the cell culture medium with the fourth composition. In various aspects, the fourth composition comprises hematopoietic factors consisting of M-CSF, GM-CSF, or FLT3 ligand, or a combination of M-CSF, GM-CSF, and FLT3 ligand.

In one embodiment, a process for generating mononuclear phagocytes from pluripotent stem cells includes (a) culturing the pluripotent stem cells in an adherent culture with a first composition comprising BMP-4 but not comprising serum; (b) culturing the cells obtained by step (a) in an adherent culture with a second composition comprising bFGF, VEGF, and SCF but not comprising serum; (c) culturing the cells obtained by step (b) in an adherent culture with a third composition comprising SCF, IL-3, thrombopoietin, M-CSF, and FLT3 ligand but not comprising serum; and (d) culturing the cells obtained by step (c) in an adherent culture, with a fourth composition comprising M-CSF, GM-CSF, and FLT3 ligand, wherein macrophages and/or monocytes are produced/collected. Alternatively, the process for generating the mononuclear phagocytes from pluripotent stem cells may be conducted in a suspension culture. For example, the step (d) may be performed in a suspension culture such as in a bioreactor with the forth composition comprising M-CSF, GM-CSF, and FLT3 ligand. Table 1 shows that cells cultivated in suspension culture in a bioreactor are alive. Culturing in a suspension culture includes collecting cells present in supernatant of the culture when cell culture medium is exchanged, and adding the collected cells back to the cell culture.

In some embodiments, the processes for the generating mononuclear phagocytes from pluripotent stem cells comprise performing one or more of the following four steps: First, contacting a cell culture with a first composition comprising BMP4 in a culture medium, wherein when the cell culture is initially contacted with the first composition the cell culture comprises pluripotent stem cells; Second, contacting the cell culture with a second composition comprising one or more of bFGF, SCF, and VEGF-A (for example each of bFGF, SCF, and VEGF-A) in a hematopoietic cell medium; Third, contacting the cell culture with a third composition comprising one or more of SCF, IL-3, TPO, M-CSF, and FLT3 ligand (for example each of SCF, IL-3, TPO, M-CSF, and FLT3 ligand) in a hematopoietic cell medium; and Fourth, contacting the cell culture with a fourth composition comprising one or more of M-CSF, FLT3 ligand, and GM-CSF (for example each of M-CSF, FLT3 ligand, and GM-CSF) in a hematopoietic cell medium, thereby generating the mononuclear phagocytes. In some embodiments, all of the above four steps are performed in order. In various embodiments, the generated mononuclear phagocytes are not further differentiated or stimulated into microglia or dendritic cells. In additional embodiments, the generated mononuclear phagocytes are not further differentiated or stimulated or macrophages; while alternatively, the generated mononuclear phagocytes may be differentiated to obtain at least some macrophages. In some of such embodiments, the medium used for any of these four steps is a serum free medium. In some of such embodiments, the medium used for any of these four steps is a chemically-defined medium. In some of such embodiments, all or any of the above four steps are performed in an extracellular matrix-coated dish or well plate. In some aspects, said extracellular matrix is a reconstituted basement membrane preparation extracted from Engelbreth-Holm-Swarm mouse sarcoma cells.

In one embodiment, a process for generating mononuclear phagocytes from pluripotent stem cells includes incubating the pluripotent stem cell in a first medium supplemented with bone morphogenetic protein 4 (BMP-4), thereby forming a first medium-treated cell; incubating the first medium-treated cell in a second medium supplemented with basic fibroblast growth factor (bFGF), vascular endothelial growth factor (VEGF), and stem cell factor (SCF), thereby forming a second medium-treated cell; incubating the second medium-treated cell in a third medium supplemented with SCF, interleukin 3 (IL-3), thrombopoietin (TPO), macrophage colony-stimulating factor (M-CSF), and FLT3 ligand, thereby forming a third medium-treated cell; and incubating the third medium-treated cell in a fourth medium supplemented with M-CSF, granulocyte-macrophage colony-stimulating factor (GM-CSF), and FLT3 ligand, thereby forming a fourth medium-treated cell, which is a mononuclear phagocyte differentiated from the pluripotent stem cell.

In one embodiment, a process for generating monocytes from pluripotent stem cells includes incubating iPSCs in a first medium supplemented with BMP-4, thereby forming a first medium-treated cell; incubating the first medium-treated cell in a second medium supplemented with bFGF, VEGF, and SCF, thereby forming a second medium-treated cell; incubating the second medium-treated cell in a third medium supplemented with SCF, IL-3, TPO, M-CSF, and FLT3 ligand, thereby forming a third medium-treated cell; and incubating the third medium-treated cell in a fourth medium supplemented with M-CSF, GM-CSF, and FLT3 ligand, thereby forming a fourth medium-treated cell, which is a monocyte differentiated from the pluripotent stem cell. The obtained monocytes generated from iPSCs are suitable for use in transplantation, transfusion, or otherwise administered to a patient in need thereof.

Some aspects provide that the first medium is a feeder-free culture medium, mTeSR, and supplemented with BMP-4. BMP-4 can be added to the first medium for a final concentration between 10 and 200 ng/mL, or between 40 and 160 ng/mL, or between 60 and 120 ng/mL, or about 80 ng/mL. In some aspects, the concentration of BMP-4 is 5 ng/mL to 150 ng/mL. In some aspects, the concentration of BMP-4 is 10 ng/mL to 100 ng/mL. In some aspects, the concentration of BMP-4 is 20 ng/mL to 80 ng/mL. In further aspects, the first medium is a standard mTeSR medium, not mTeSR custom medium; and therefore the first medium is mTeSR that contains bFGF, TGFβ, GABA, pipecolic acid, and lithium chloride. This first medium with the supplement can be used, in one or more fresh volumes, to cultivate the stem cell for about 3 days, or from day 1 to day 4. In some aspects, a tissue culture medium suitable for maintenance of stem cells is used as the first medium. In other aspects, tissue culture medium suitable for differentiation of stem cells is used as the first medium.

“TeSR” is a serum-free, xeno-free medium shown to support derivation and long-term feeder-independent culture of hPSCs, and was developed by Tenneille Ludwig and colleagues (Ludwig T E et al., Nat Biotechnol. 24:185-7, 2006). The formulation of “TeSR” included high levels of bFGF, together with TGF, GABA, pipecolic acid, and lithium chloride. This original publication by Ludwig et al., described the use of cell support matrix composed of four human components (collagen IV, fibronectin, laminin, and vitronectin). Ludwig and colleagues further developed modifications to the medium (“mTeSR1”), which does include some animal-sourced proteins yet retains the advantages of being fully-defined and serum-free and supports the self-renewal of hPSCs without requiring feeder cells (Ludwig T E, et al., Nat Methods 3:637-46, 2006). A mTeSR1 medium, according to Ludwig T E, et al., Nat Methods 3:637-46, 2006, contains: DMEM/F12, Stock B (including dissolved bovine serum albumin, thiamine, reduced glutathione, L-ascorbic acid 2-phosphate magnesium salt, selenium, Trace Elements B, Trace Elements C, insulin, holo-transferrin), zebrafish bFGF, TGFβ1, pipecolic acid, GABA, lithium chloride, lipid, L-glutamine-β mercaptoethanol, MEM NEAA, and NaHCO₃, which upon mixing is adjusted for pH to be 7.4 using NaOH and for osmolarity to be between 340 and 350 mOsMol using crystalline NaCl, and preferably filter-sterilized before use.

Some aspects provide that the second medium is a serum-free medium, e.g., StemPro-34, and supplemented with (1) bFGF at a final concentration between 5 and 100 ng/mL, or between 10 and 50 ng/mL, or between 20 ng/mL and 35 ng/mL, or about 25 ng/mL; (2) VEGF at a final concentration between about 10 and 200 ng/mL, between about 40 and 120 ng/mL, between about 60 and 100 ng/mL, or about 80 ng/mL; and (3) SCF at a final concentration between about 10 and 500 ng/mL, or between 30 and 300 ng/mL, or between 50 and 150 ng/mL, or between 80 and 120 ng/mL, or about 100 ng/mL. This second medium with the supplements can be used, in one or more fresh volumes, to cultivate the cells for about 2 days, from day 4 to day 6.

Some aspects provide that the third medium is a serum-free medium, e.g., StemPro-34, and supplemented with (1) SCF at a final concentration between 5 and 100 ng/mL, or between 25 and 75 ng/mL, or between 40 and 60 ng/mL, or about 50 ng/mL; (2) IL-3 at a final concentration between 5 and 100 ng/mL, or between 25 and 75 ng/mL, or between 40 and 60 ng/mL, or about 50 ng/mL; (3) TPO at a final concentration between 0.5 and 20 ng/mL, or between 1 and 10 ng/mL, or between 3 and 7 ng/mL, or about 5 ng/mL; (4) M-CSF at a final concentration between 5 and 100 ng/mL, or between 25 and 75 ng/mL, or between 40 and 60 ng/mL, or about 50 ng/mL; and (5) FLT3 (or FLT3 ligand) at a final concentration between 5 and 100 ng/mL, or between 25 and 75 ng/mL, or between 40 and 60 ng/mL, or about 50 ng/mL. This third medium with the supplements can be used, in one or more fresh volumes, to cultivate the cells for about 6 or 7 days, e.g., from day 6 to day 12 or day 13.

Some aspects provide that the fourth medium is a serum-free medium, e.g., StemPro-34, and supplemented with (1) M-CSF at a final concentration between 5 and 100 ng/mL, or between 25 and 75 ng/mL, or between 40 and 60 ng/mL, or about 50 ng/mL; (2) GM-C SF at a final concentration between about 5 and 50 ng/mL, or between 10 and 40 ng/mL, or between 20 and 30 ng/mL, or about 25 ng/mL; and (3) FLT3 (or FLT3 ligand) at a final concentration between 5 and 100 ng/mL, or between 25 and 75 ng/mL, or between 40 and 60 ng/mL, or about 50 ng/mL.

Additional aspects provide that in stages 2-4, any suitable hematopoietic cell medium can be used as the second, third, and fourth mediums. In one embodiment, the hematopoietic cell medium is “StemPro-34.” The composition of StemPro-34 medium is known in the art and described in, for example, EP 0891419 (or US20040072349, US20100297090) entitled “Hematopoietic Cell Culture Nutrient Supplement” and WO1997033978 (or US20040072349, US20100297090), the contents of which are hereby incorporated by reference. However, one of skill in the art will recognize that there are several other types of media that are equivalent to StemPro-34 medium in terms of their suitability for use in culturing hematopoietic cells—any of which could be used.

In various instances, the concentration of the cytokine or the like, including the hematopoietic factor, to be used in each step is not restricted as long as the cells of interest can be obtained at the concentration. In some aspects, the concentration of bFGF in the cell culture medium in respective step is 10 ng/mL to 100 ng/mL. In some aspects, the concentration of bFGF in the cell culture medium in respective step is 20 ng/mL to 50 ng/mL. In some aspects, the concentration of bFGF in the cell culture medium in respective step is about 25 ng/mL. In some aspects, the concentration of VEGF in the cell culture medium in respective step is 20 ng/mL to 100 ng/mL. In some aspects, the concentration of VEGF in the cell culture medium in respective step is 30 ng/mL to 70 ng/mL. In some aspects, the concentration of VEGF in the cell culture medium in respective step is about 50 ng/mL. In some aspects, the concentration of SCF in the cell culture medium in respective step is 20 ng/mL to 100 ng/mL. In some aspects, the concentration of SCF in the cell culture medium in respective step is 30 ng/mL to 70 ng/mL. In some aspects, the concentration of SCF in the cell culture medium in respective step is about 50 ng/mL. In the case of IL-3, the concentration is 5 ng/mL to 100 ng/mL in some instances. The concentration of IL-3 may in some aspects be 30 ng/ml to 70 ng/ml. In other aspects, the concentration of IL-3 may be about 50 ng/ml. In the case of TPO, the concentration is 1 ng/mL to 25 ng/mL. In some aspects, the concentration of TPO is preferably 1 ng/mL to 10 ng/mL. In some aspects, the concentration of TPO is about 5 ng/mL. In the case of Flt3-ligand (FLT3L), the concentration is 10 ng/ml to 100 ng/ml in various aspects. In some aspects, the concentration of FLT3L is 30 ng/ml to 70 ng/ml. In some aspects, the concentration of FLT3L is about 50 ng/ml. In the case of GM-CSF, the concentration is 5 ng/ml to 100 ng/ml in various aspects. In some aspects, the concentration of GM-C SF is preferably 10 ng/ml to 50 ng/ml. In some aspects, the concentration of GM-CSF is about 25 ng/ml. In the case of M-CSF, the concentration is 5 ng/ml to 100 ng/ml in various aspects. In some aspects, the concentration of M-CSF is preferably 30 ng/ml to 70 ng/ml, or more preferably 50 ng/ml.

In various aspects, the process of generating mononuclear phagocytes from pluripotent stem cells (preferably from iPSCs) include using respective factors at the following combination:
BMP-4 is 60 ng/mL to 100 ng/mL in the first cell culture medium;
bFGF is 15 ng/mL to 30 ng/mL, VEGF is 60 ng/mL to 100 ng/mL, and SCF is 80 ng/mL to 120 ng/mL, in the second cell culture medium;
SCF is 40 ng/mL to 60 ng/mL, IL-3 is 40 ng/mL to 60 ng/mL, TPO is 4 ng/mL to 6 ng/mL, M-CSF is 40 ng/mL to 60 ng/mL, and FLT3L is 40 ng/mL to 60 ng/mL, in the third cell culture medium; and
M-CSF is 40 ng/mL to 60 ng/mL, GM-CSF is 15 ng/mL to 30 ng/mL, and FLT3L is 40 ng/mL to 60 ng/mL, in the fourth cell culture medium.

Various embodiments also provide that, in terms of the period of each step, the above Step (a) (or “Stage 1”) is performed for not less than 2 days, preferably for not less than 2 days and not more than 6 days, more preferably for 4 days. The above Step (b) (or “Stage 2”) is performed for not less than 1 day, preferably for not less than 1 day and not more than 5 days, more preferably for 2 days. The above Step (c) (or “Stage 3”) is performed for not less than 5 days, preferably not less than 6 days and not more than 14 days, more preferably 9 days. The above Step (d) (or “Stage 4”) is performed for not less than 3 days, preferably not less than 3 days and not more than 90 days. In some embodiments, Step (d) (or “Stage 4”) is performed for at least 55 days or 60 days, and up to about 90 days.

In some embodiments, the generated mononuclear phagocytes are further cultured in a bioreactor, so as to grow to a clinically relevant number in the order of at least 1×10⁶ cells. In some embodiments, the process of generating mononuclear phagocytes from pluripotent stem cells are performed in a bioreactor, starting from any one of Step (a) (“stage 1”), Step (b) (“stage 2”), Step (c) (“stage 3”), or Step (d) (“stage 4”). As demonstrated in Example 3, mononuclear phagocytes generated from the pluripotent stem cells and proliferated in a bioreactor (for various days from 1-10 days, 11-20 days, 21-30 days, 31-40 days, 41-50 days, 50-60 days, or longer) show similar gene expression profiles and monocyte/macrophage marker expression levels to those generated in a well-plate.

Bioreactors known in the art are generally suitable for growing iMPs to obtain clinically relevant numbers for administration. Exemplary bioreactors include stirred flasks, also called stirrer tank bioreactors, in which impeller mixing maintains the cells in suspension and the fluid movement helps in mass transport of nutrients and wastes. In addition to stirred flasks, we also conceive using a rocker bag system and/or a G-REX® system for the scale-up production of iMPs. For example, a rocker bag system includes a rocker (including a base and providing a platform, such as in the shape of a tray, optionally further including a heater and/or thermocouple) and one or more cell culture rocker bags (for enclosing cell cultures, and suitable for placement on the platform). The rocker produces a smooth-rocking, wave motion that provides for gentle, efficient mixing and gas transfer. Cell culture rocker bags usually contains ports for importing and exporting fluid and/or air in and out of the bags. G-REX® refers to gas permeable rapid expansion. A G-REX bioreactor gives cells unlimited and undisturbed access to nutrients and oxygen to produce a large quantity of cells, eliminating media exchanges and the complex hardware required in integrated systems.

In various embodiments, the myelomonocytic cells or mononuclear phagocytes generated from pluripotent stem cells (e.g., iPSCs) in the process disclosed herein are positive for monocyte/macrophage markers such as CD14, CD16, CD64, CD11b, CD11c, CD71, thereby termed as iMPs (mononuclear phagocytes generated from iPSCs), or in various instances including monocytes generated from iPSCs and/or macrophages generated from iPSC (termed as iMACs in priority application U.S. 63/234,984), and which no longer or has little expression of hematopoietic stem cell marker CD34. In various embodiments, the mononuclear phagocytes generated from stem cells (e.g., from iPSCs) are not microglia, as the method of differentiation does not include cultivating any of the generated cells in a Microglial Medium. Differentiated mononuclear phagocytes can be cultivated in fresh volumes of the four medium (with the supplements) until harvest. In various embodiments, the method of differentiation further includes, or is accompanied by, amplifying the cells by replenishing fresh volumes of the medium at respective stage, and optionally passaging the cells. In some implementations, harvested cells are directly administered to a subject, optionally with some dilution or concentration. In various embodiments, at least 50%, 60%, 70%, 80%, or 90% of the harvested cells from the fourth medium-treated cells are monocytes. Preferably at least 50% of cells harvested after the fourth medium are monocytes. More preferably at least 70% or about 70% of the cells harvested after the fourth medium are monocytes. Cells can be analyzed using antibodies targeting antigens such as CD34, CD11b, CD11c, CD14, and CD16 to determine the percentage of cells that express monocyte/macrophage markers.

In other implementations, harvested mononuclear phagocytes derived from stem cells are cryopreserved, to maintain product stability during storage and shipping steps. In some aspects, the harvested mononuclear phagocytes generated from the process are purified, for example, by marker of CD14. The purification of CD14-positive cells can be performed by a method well known to those skilled in the art, and the method is not restricted. For example, the cells can be purified using CD14 MicroBeads or flow cytometer. In some aspects, the mononuclear phagocytes are harvested without further sorting by one or more markers, especially not sorted by marker C3CR1.

In various embodiments, a significant amount of mononuclear phagocytes generated from pluripotent stem cells can be cultured in a bioreactor, e.g., achieving a clinically meaningful amount in the order of at least 1×10⁶, 1×10⁷, or 1×10⁸ (per dose in a therapy containing two or more doses, or per therapy), and preferably the mononuclear phagocytes cultured in a bioreactor maintain the genetic profiles (including expression amounts of macrophage markers or monocyte/macrophage markers) over a period of time of at least 5 days, 10 days, 2 weeks, 3 weeks, 4 weeks, or longer. In some embodiments, the mononuclear phagocytes generated from pluripotent stem cells, especially in a clinically meaningful amount through culturing in a bioreactor, after a period of time (e.g., 1 week, 2 weeks, 3 weeks, 1 month, 2 months, 3 months, or longer) in bioreactor cultivation or in frozen storage, maintain the genetic profiles (including expression amounts of monocyte/macrophage markers) at levels at least 95%, 90%, 85%, 80%, 75%, 70%, 65%, 60%, or 50%, compared to freshly generated mononuclear phagocytes from the pluripotent stem cells (which may be collected within 7 days of “stage 4” differentiation).

Notably, in various embodiments, the generated iMPs have differential gene expression compared to naturally occurring monocytes or naturally occurring macrophages or naturally occurring mononuclear phagocytes. The generated iMPs may be positive for similar markers and behave similarly in function tests especially in vivo, as the naturally occurring counterparts, thereby having the therapeutic efficacy as demonstrated in Examples. Neither the iPSCs nor the naturally occurring macrophages/monocytes are proliferative, but as detailed in Examples 2 and 3, the invention provides cysts differentiated from iPSCs, so that iMPs can bud off from the cysts and thereby be expanded in production to therapeutic quantities (or clinically meaning quantities).

In various embodiments of the methods, the composition comprising a population of mononuclear phagocytes are administered in two or more exposures to the subject. In one embodiment, the composition comprising a population of mononuclear phagocytes is administered at least for at least 3 doses to the subject. In one embodiment, the composition comprising a population of the mononuclear phagocytes generated from iPSCs is administered for 4-10 doses to the subject. In one embodiment, the composition comprising a population of the mononuclear phagocytes generated from iPSCs is administered for 6-12 doses to the subject. In some implementations, the composition is administered on a weekly, biweekly, bimonthly, or monthly basis, or as needed by the subject. In some implementations, the composition in each exposure to the subject can include at least 10⁶ cells, 10⁷ cells, 10⁸ cells, 10⁹ cells, 1¹⁰ cells, or 10¹¹ cells. In various implementations, the therapeutically effective dose of cells depends on a patient's needs, age, physiological condition and healthy state, and the tissue size of to be reached and therapeutic goal, implant site, pathology degree (deterioration of neurons level), selected mode of movement and therapeutic strategy. In some implementations, a low dose of cells is repeatedly transplanted. These cells can be used for the treatment of neural acute or chronic injury, and/or delaying the onset of, alleviating, or treating a neurodegenerative disease and neuronal disease.

Various embodiments of the treatment methods are for an aging mammal, e.g., a human at an age of at least 50 years old, at least 60 years old, at least 70 years old, at least 80 years old, or at least 90 years old.

In other embodiments, the methods disclosed herein are for a subject developing or diagnosed with a neurodegenerative disease, such as Alzheimer's disease. In further embodiments, the methods disclosed herein are for a subject first exhibiting pathology of Alzheimer's disease, such as when amyloid and microglial activation is detected. In yet other embodiments, the methods disclosed herein are for a subject with suffering significantly of Alzheimer's disease or having been diagnosed with Alzheimer's disease for at least six months, 1 year, 2 years or longer, and the methods alleviate or reverse the pathology. In certain embodiments, a neurodegenerative disease or neuronal disease of the method is selected from Parkinson's disease, Alzheimer's disease, amyotrophic lateral sclerosis (ALS), multiple sclerosis, Rett syndrome, diffuse leukoenchephalopathy with spheroids, hereditary diffuse leukoenchephalopathy with axonal spheroids, frontotemporal lobar degeneration (FTLD), familial FTLD, schizophrenia, autism spectrum disorders, Huntington Chorea, dementia with Lewy body, cerebellar ataxia, stein-leventhal syndrome, Spinal injury, epilepsy, the group that apoplexy becomes with local ischemia group.

In additional embodiments, the methods disclosed herein are for a subject with an Alzheimer's disease (e.g., exhibiting signs or symptoms, or diagnosed with, Alzheimer's disease), but the subject does not have amyotrophic lateral sclerosis (ALS).

In additional embodiments, the methods disclosed herein are for a subject having a deficiency in macrophages or a disease or disorder associated with a defect or deficiency in macrophages, so that administering the mononuclear phagocytes generated from iPSCs may produce macrophages in the subject after the administration.

Various embodiments provide that the methods disclosed herein further include selecting a subject having, showing signs of, or is at risk of developing, a neurodegenerative disease for receiving the administration of the mononuclear phagocytes generated from pluripotent stem cells. Various embodiments provide that the methods disclosed herein further include obtaining autologous somatic cells (e.g., fibroblasts, blood cells) from a subject having, showing signs of, or is at risk of developing, a neurodegenerative disease, then generating iPS cells from the autologous somatic cells by a reprogramming process known in the art, so as to obtain mononuclear phagocytes generated from the iPS cells for administration to the subject. Additional embodiments provide that the methods disclosed herein further include growing the generated mononuclear phagocytes in a bioreactor to obtain at least 1×10⁶, or 1×10⁷ cells,

Additional embodiments provide methods for generating mononuclear phagocytes (or myeloid monocytic cells, or myeloid lineage cells) from pluripotent stem cells, wherein the methods include the steps of:
incubating the stem cell in a first medium supplemented with bone morphogenetic protein 4 (BMP-4), thereby forming a first medium-treated cell;
incubating the first medium-treated cell in a second medium supplemented with basic fibroblast growth factor (bFGF), vascular endothelial growth factor (VEGF), and stem cell factor (SCF), thereby forming a second medium-treated cell;
incubating the second medium-treated cell in a third medium supplemented with SCF, interleukin 3 (IL-3), thrombopoietin, macrophage colony-stimulating factor (M-CSF), and FLT3 ligand, thereby forming a third medium-treated cell; and
incubating the third medium-treated cell in a fourth medium supplemented with M-CSF, granulocyte-macrophage colony-stimulating factor (GM-CSF), and FLT3 ligand, thereby forming a fourth medium-treated cell, which is a macrophage or monocyte differentiated from the stem cell,
wherein the methods do not include culturing in the presence of IL-34, or a combination of IL-34 and GM-CSF, or IL-4, or a combination of IL-4 and GM-CSF, or a combination of M-CSF and IFN-gamma, or a combination of M-CSF and IL-4.

In some aspects of the methods for differentiation, the methods do not include incubating the fourth medium-treated cell in a microglial differentiation medium or a dendritic cell differentiation medium. In some aspects, at least 50% of the fourth medium-treated cells are monocytes; or the cells obtained from Step (d) (or “stage 4”) are substantially pure mononuclear phagocytes (which include monocytes and macrophages), characterized for expression of markers CD14, CD16, CD64, CD11b, CD11c, and CD71. In some aspects of the methods for differentiation, the generated mononuclear phagocyte is not a microglia, not a dendritic cell, and the generated mononuclear phagocyte is positive for one or more markers of CD11b, CD11c, CD14, and CD16.

In some aspects, the mononuclear phagocyte is differentiated from an iPSC prepared by reprogramming blood cells, preferably peripheral blood mononuclear cells (PBMCs), from a subject, e.g., from a healthy human subject, a young human (e.g., a human at an age within the age group of 5-11, 12-16, 17-18, 19-21, 22-34, or 35-49 years old), or a young healthy human subject. In further embodiments, the mononuclear phagocyte is differentiated from an iPSC prepared by reprogramming fibroblasts obtained from the subject.

In some embodiments, methods for reprogramming blood cells to iPSCs are disclosed in WO2017219000, U.S. Pat. Nos. 10,221,395, and 10,745,671, which are incorporated by reference herein. For example, a method of generating blood cell derived iPSC comprises: delivering a quantity of EBNA1 and reprogramming factors comprising Oct-4, Sox-2, Klf-4, 1-Myc, Lin-28, SV40 Large T Antigen (“SV40LT”), and short hairpin RNAs targeting p53 (“shRNA-p53”) into a quantity of blood cells; and culturing the blood cells in a reprogramming media for at least 4 days, wherein delivering the EBNA1 and reprogramming factors and culturing in a reprogramming media generates blood cell derived induced pluripotent stem cells, wherein the reprogramming factors are encoded in four oriP/EBNA1 derived vectors comprising a first vector encoding Oct4, Sox2, SV40LT and Klf4, a second vector encoding Oct4 and shRNA-p53, a third vector encoding Sox2 and Klf4, and a fourth vector encoding 1-Myc and Lin-28; and wherein a fifth oriP/EBNA1 derived vector encodes EBNA.

In some aspects, the mononuclear phagocyte is differentiated from a stem cell or induced pluripotent stem cell from a species, and used in treating a subject of the same species. In some aspects, the mononuclear phagocyte is differentiated from a stem cell or an induced pluripotent stem cell from a species at a young age, e.g., said stem cell obtained from or reprogrammed from a somatic cell obtained from a subject of a species at an age younger than the first half of the average life span of the species. In some aspects, the mononuclear phagocyte is differentiated from a mouse stem cell obtained from a mouse of less than 4 months old, e.g., between 3-4 months old, between 2-3 months old, between 1-2 months old. In some aspects, the mononuclear phagocyte is generated from iPSCs reprogrammed from somatic cells of a human in his/her early teens, twenties, or thirties, or forties years old, and used when the human exhibits aging or a neurodegenerative disease or disorder. In another aspect, the mononuclear phagocytes is differentiated from a human subject with a cognitive impairment or a neurodegenerative disease/disorder, or from an aged human subject (e.g., at least 40 years old, at least 50 years old, at least 60 years old, at least 70 years old, or at least 80 years old). In another aspect, the mononuclear phagocyte is differentiated from an aged mouse, e.g., about 11-13 months old.

In various embodiments, the present invention provides a pharmaceutical composition. The pharmaceutical composition includes a population of mononuclear phagocytes which are derived from a stem cell, e.g., differentiated from induced pluripotent stem cell. In some embodiments, a patient's own (autologous) cells are used to derive the mononuclear phagocytes. In other embodiments, donated (allogenic) cells are used to derive mononuclear phagocytes. Mononuclear phagocytes differentiated from stem cells can be maintained in liquid suspensions or formulations until administration.

The disclosed methods can improve cognitive functions and/or neural health. For example, a treated subject can have an improved spatial working memory and/or an improved short-term memory, compared to the subject's condition before the treatment. A treated subject can also have an increased level of synaptic transporter, increased microglia level, and/or increased astrocyte level, compared to a control. In some aspects, the control can be a subject with neurodegenerative disorder not treated with the cell therapy disclosed herein. In other aspects, the control can be a baseline level of the subject before the treatment. Alternatively, the treated subject can exhibit a comparable cognitive function or neural health as a young and/or healthy subject.

Mononuclear phagocytes differentiated from stem cells by a differentiation method disclosed herein are also provided. In various aspects, monocytes generated from iPSCs by a process disclosed herein are provided. In various implementations, the mononuclear phagocytes differentiated from induced pluripotent stem cells are provided in a composition, or a pharmaceutical composition, with one or more excipients.

Preferably, the mononuclear phagocytes are differentiated from autologous stem cells of a subject to whom the generated mononuclear phagocytes, often after expansion, will be administered to. For example, blood cells or fibroblasts or another somatic cell from the mammal are reprogrammed to induced pluripotent stem cells, which are then differentiated into the mononuclear phagocytes by a differentiation method disclosed herein; and the obtained mononuclear phagocytes are infused/transplanted or otherwise injected to said mammal, who is in need of cognitive function improvement or suffers from a neurodegenerative disorder. In other examples, somatic cells of a healthy or young mammal are reprogrammed to induced pluripotent stem cells; and the obtained mononuclear phagocytes are infused, transplanted or injected to a mammal in need of cognitive function improvement or suffers from a neurodegenerative disorder. In one embodiment, the multipotential stem cell is mouse's, pig's, monkey's, sheep's, or a human being's embryonic stem cell. In another embodiment, the experimenter is patient, more preferably human patients, and the multipotential stem cell is reprogrammed from the human patient's own tissue cells. Additional procedures for reprogramming somatic cells into induced pluripotent stem cells (or multipotential stem cells) are known, for example, as described in Zhao et al., iScience 23, 101192, 2020 and in U.S. Pat. Nos. 9,534,205, 9,394,524, 9,540615, and 9,771,563, which are herein incorporated by reference.

Some embodiments provide a method for reducing inflammation in a subject or treating a subject with an inflammation associated disease, comprising administering to the subject a therapeutically effective amount of a pharmaceutical composition comprising the mononuclear phagocytes generated from iPSCs, wherein the mononuclear phagocytes are generated from the iPSCs by a process that comprises or consists essentially of:
incubating the iPSCs in a first medium supplemented with bone morphogenetic protein 4 (BMP-4), thereby forming a first medium-treated cell;
incubating the first medium-treated cell in a second medium supplemented with basic fibroblast growth factor (bFGF), vascular endothelial growth factor (VEGF), and stem cell factor (SCF), thereby forming a second medium-treated cell;
incubating the second medium-treated cell in a third medium supplemented with SCF, interleukin 3 (IL-3), thrombopoietin, macrophage colony-stimulating factor (M-CSF), and FLT3 ligand, thereby forming a third medium-treated cell; and
incubating the third medium-treated cell in a fourth medium supplemented with M-CSF, granulocyte-macrophage colony-stimulating factor (GM-CSF), and FLT3 ligand, thereby forming a fourth medium-treated cell, which is a mononuclear phagocyte differentiated from the iPSC.

Some embodiments provide a method for improving cognitive function in a subject, or treating a subject with a neurodegenerative disorder, or alleviating, treating, or delaying onset of a neurodegenerative disorder in a subject, wherein the methods include administering to the subject a therapeutically effective amount of a pharmaceutical composition comprising mononuclear phagocytes generated from iPSCs, wherein the mononuclear phagocytes are differentiated from the iPSCs by a process that comprises or consists essentially of:
incubating the iPSCs in a first medium supplemented with bone morphogenetic protein 4 (BMP-4), thereby forming a first medium-treated cell;
incubating the first medium-treated cell in a second medium supplemented with basic fibroblast growth factor (bFGF), vascular endothelial growth factor (VEGF), and stem cell factor (SCF), thereby forming a second medium-treated cell;
incubating the second medium-treated cell in a third medium supplemented with SCF, interleukin 3 (IL-3), thrombopoietin, macrophage colony-stimulating factor (M-CSF), and FLT3 ligand, thereby forming a third medium-treated cell; and
incubating the third medium-treated cell in a fourth medium supplemented with M-CSF, granulocyte-macrophage colony-stimulating factor (GM-CSF), and FLT3 ligand, thereby forming a fourth medium-treated cell, which is a mononuclear phagocyte differentiated from the iPSC.

In some embodiments, the subject is an aged human being. In some embodiments, the subject has Alzheimer's disease and/or amyotrophic lateral sclerosis. In some embodiments, the pharmaceutical composition comprising the iMPs is administered intravenously. In some embodiments, the pharmaceutical composition comprising the iMPs is administered via intraperitoneal injection. In some embodiments, the methods include further performing one or more of behavioral assays (learning and memory study), neural health examination and measuring inflammation level. In some embodiments, following the administration of the pharmaceutical composition comprising the mononuclear phagocytes differentiated from stem cells, the subject exhibits an improved neural healthy, cognitive function, as assayed by one or more behavioral studies, and/or a reduced inflammation level relative to the subject's baseline prior to the treatment.

In various aspects, one or more methods disclosed herein results in an improved cognitive function (e.g., characterized in one or more behavior tests) or improved level of synaptic transport (such as VGLUT1) compared to a control subject who has the neurodegenerative disorder but has not received a treatment with the macrophages or monocytes.

In other aspects, one or more methods disclosed herein results in an improved cognitive function (e.g., characterized in one or more behavior tests) or improved level of synaptic transport (such as VGLUT1) compared to a control level which is the baseline level of the subject before receiving the treatment with the macrophages and/or monocytes generated from pluripotent stem cells.

The pharmaceutical compositions can contain a pharmaceutically acceptable excipient or carrier, such as buffers, salts, polymers, proteins, and preservatives which are added to stabilize the cells or to provide physiological osmolality. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid. The final harvest of cells prior to formulation and patient use may also carry residual amounts of cell culture supplements. Therefore, a composition disclosed herein may include excipients, which refer to components used in the formulation and to ancillary materials (e.g., cell culture supplements) that may remain in the final product. Examples of excipients include but are not limited to human serum albumin, dimethyl sulfoxide (DMSO), calcium chloride, potassium chloride, sodium chloride, sodium lactate, water, dextran and combinations thereof. “Pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid filler, diluent, excipient, solvent, or encapsulating material, or a combination thereof. The carrier is suitable for use in contact with any tissues or organs with which it may come in contact, meaning that it must not carry a risk of toxicity, irritation, allergic response, immunogenicity, or any other complication that excessively outweighs its therapeutic benefits.

In various embodiments, the pharmaceutical compositions according to the invention may be formulated for delivery via any route of administration. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection. Typically, the compositions are administered by injection. Methods for these administrations are known to one skilled in the art.

In further embodiments, the treatment and/or prophylactic methods disclosed further include administering to the subject one or more drugs, or standard therapies, to the subject having the neurodegenerative disease, such as Alzheimer's disease. In some implementations, the pharmaceutical compositions of the invention are administered concurrently with the one or more drugs or standard therapies. In some implementations, the pharmaceutical compositions of the invention are administered separately from the one or more drugs or standard (currently approved) therapies. Suitable drugs or currently approved therapies include galantamine, rivastigmine, donepezil, memantine, aducanumab (a human antibody targeting aggregated forms of amyloid-β).

Additional embodiments provide for methods of drug screening using mononuclear phagocytes generated as described herein, or as produced using the methods described herein, including but not limited to high-throughput screening methods. For example, in one embodiment the present invention provides a method of identifying a compound useful in the treatment or prevention of a disease or disorder associated with a defect in or deficiency of monocytes and/or macrophages, the method comprising: contacting a mononuclear phagocyte generated by a method disclosed herein with a candidate compound, and determining whether the candidate compound improves the defect in or deficiency of monocytes or macrophages. In some embodiments, the method for identifying the compound is a high-throughput one. In some embodiments, the identified compound is useful in the treatment or prevention of the disease or disorder in a human or a mammalian. In some embodiments, the mononuclear phagocyte is autologous or generated from autologous cells including iPSCs reprogrammed from autologous somatic cells. In some embodiments, the mononuclear phagocyte is allogeneic or generated from allogeneic cells including iPSCs reprogrammed from allogeneic cells. In some embodiments, the disease or disorder associated with a defect in, or deficiency of macrophages and/or monocytes is Alzheimer's disease. In some embodiments, the disease or disorder associated with a defect in, or deficiency of macrophages and/or monocytes is Parkinson's disease.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

The use of blood, plasma, or bone marrow from young subjects to restore cognitive function in aged subjects have significant practical drawbacks that limit their potential therapeutic value. Induced pluripotent stem cells (iPSCs) offer the ability to generate an autologous therapy.

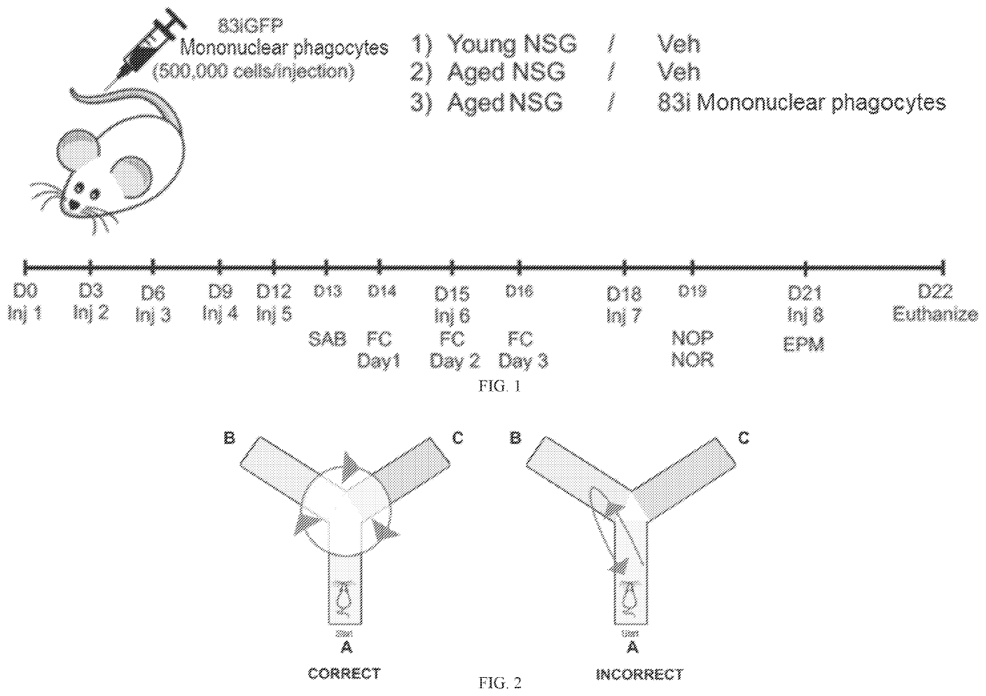

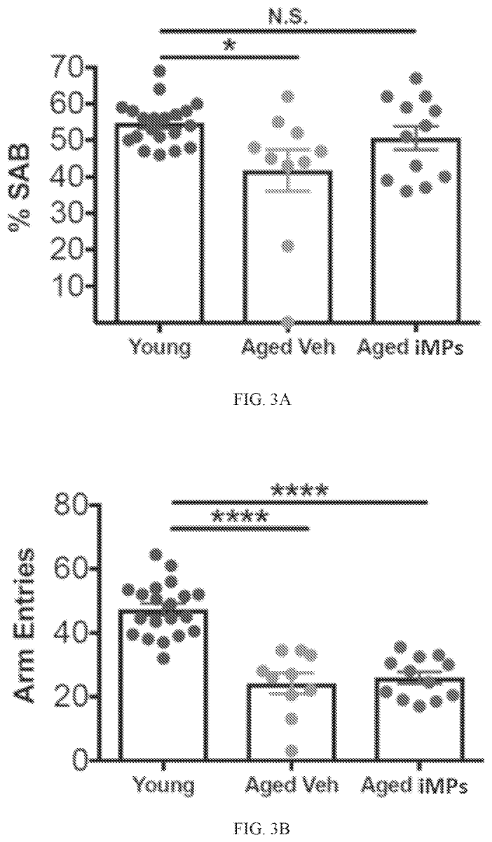

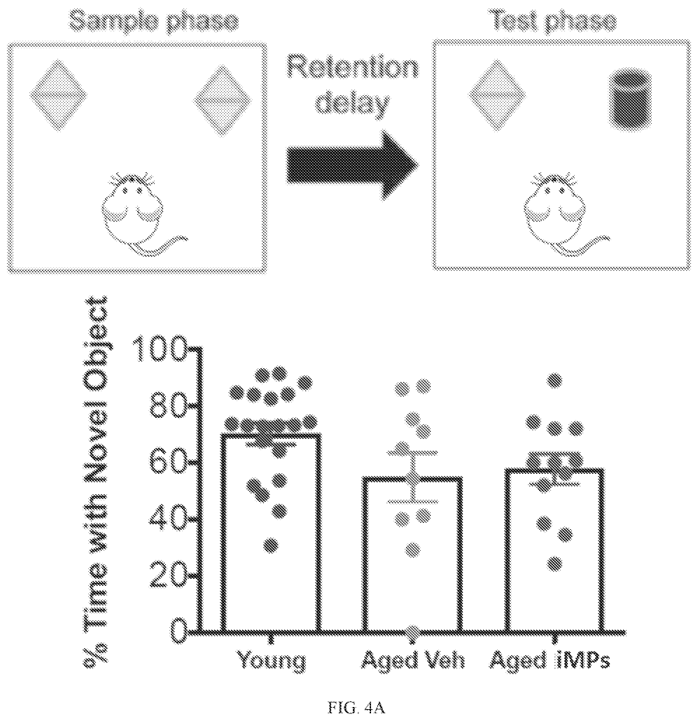

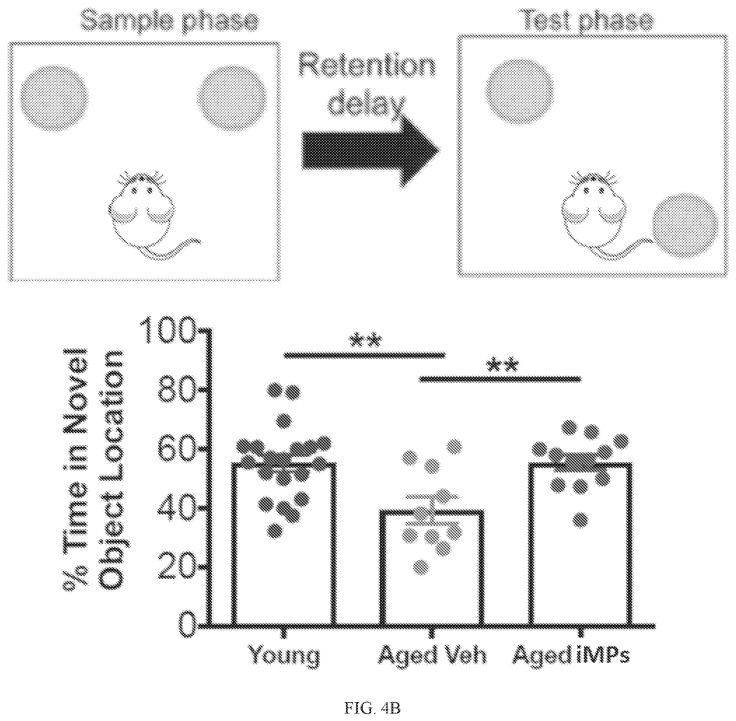

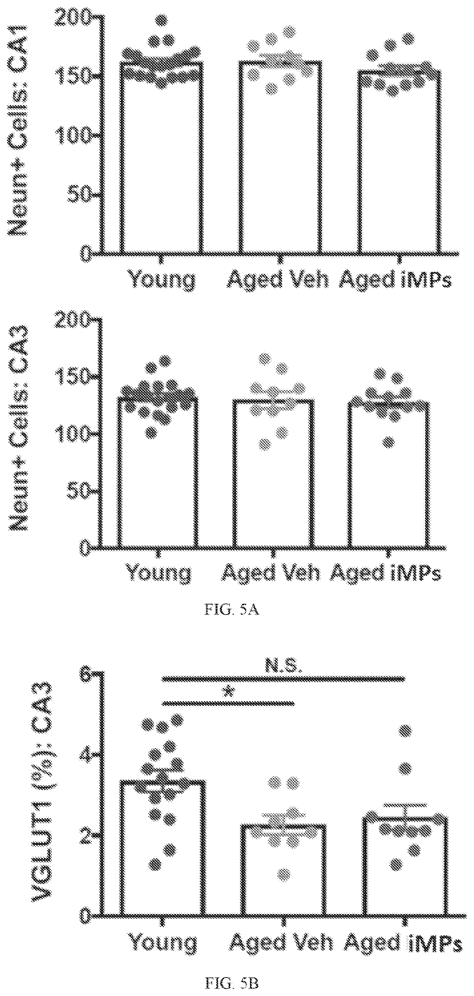

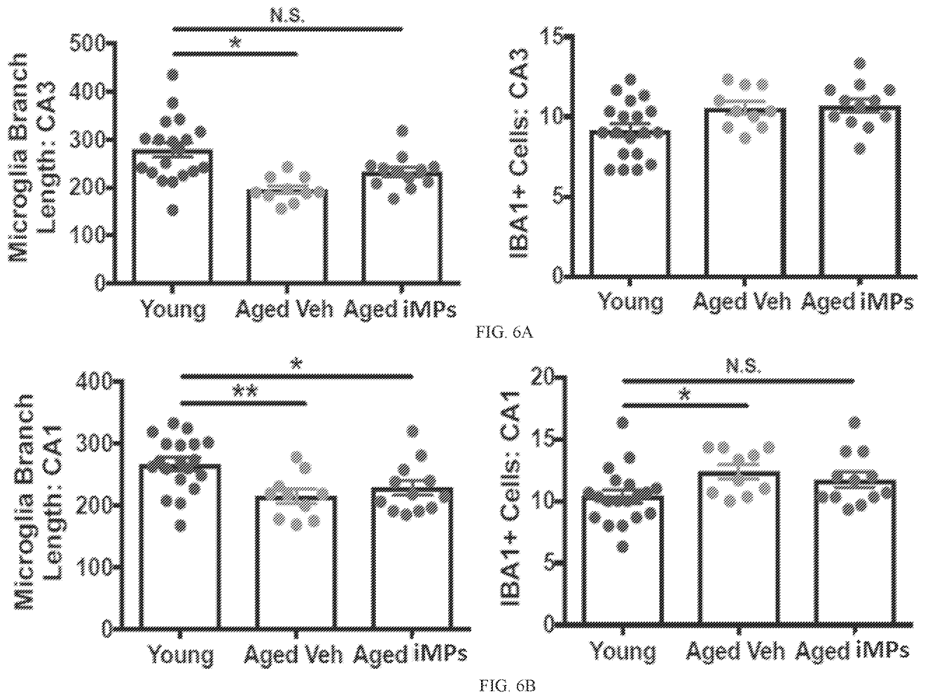

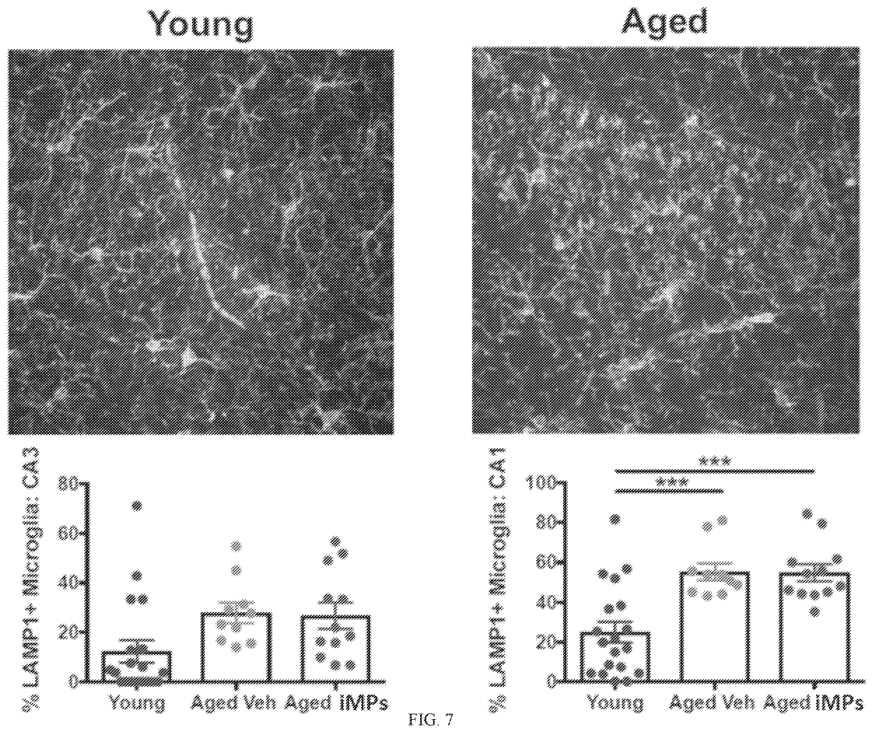

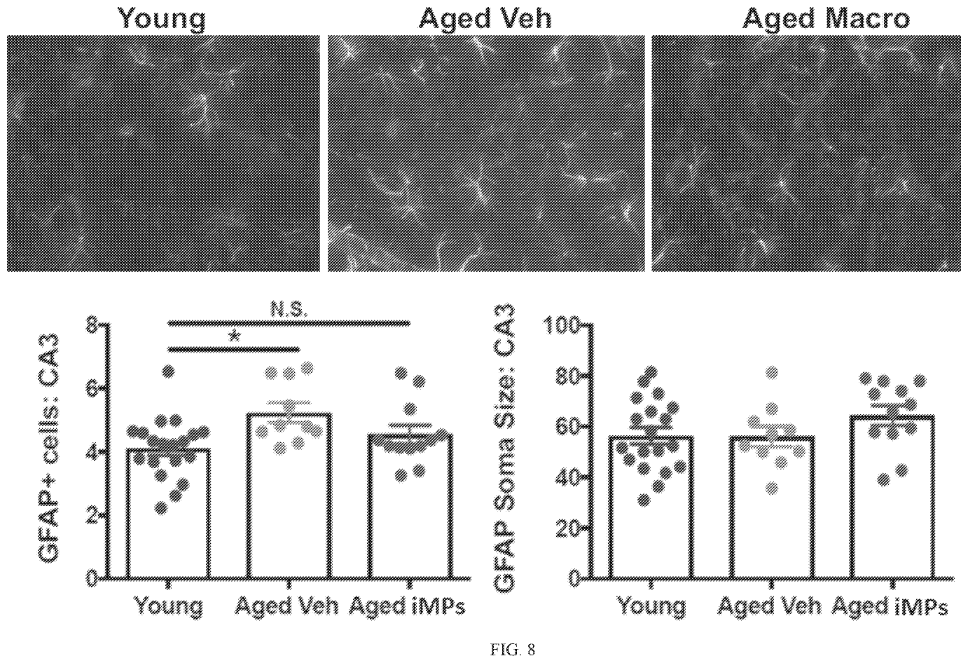

Here, we sought to identify the cell type responsible for the beneficial effects observed in studies using young plasma and bone marrow, and we generated mononuclear phagocytes from iPSCs (iMPs), which express low levels of the hematopoietic stem cell marker CD34, and high levels of the monocyte/macrophage markers CD11b, CD14, and CD16; and we administered them to aged, genetically immunocompromised NOD-scid-gamma (NSG) mice via tail vein injection. (Young mice are 3-4 months old; aged mice are 11-13 months old.) Mice received treatments every third day for 22 days (FIG. 1) and were tested on a number of behavioral assays. We found significant improvements in spatial working memory in the spontaneous alternation test (FIG. 3A, 3B) as well as in hippocampal-dependent short-term memory in the novel object placement assay (FIG. 4B). Furthermore, treatment with iMPs had significant effects on several key neuronal health indices including the synaptic transporter, VGLUT1, (FIG. 5B), which is decreased in aged mice but restored with treatment; and on microglial and astrocytic numbers and morphology (FIGS. 6A, 6B, and 8). The increased numbers of astrocyte and microglia as well as the decrease in microglial branch length seen in aged animals are all reversed by treatment with iMPs. These findings indicate that immune cells, specifically monocytes and macrophages, may be responsible for the regenerative effects previously observed after young plasma transfusions or bone marrow transplants. Importantly, we find that iMPs have significant regenerative potential in aging.

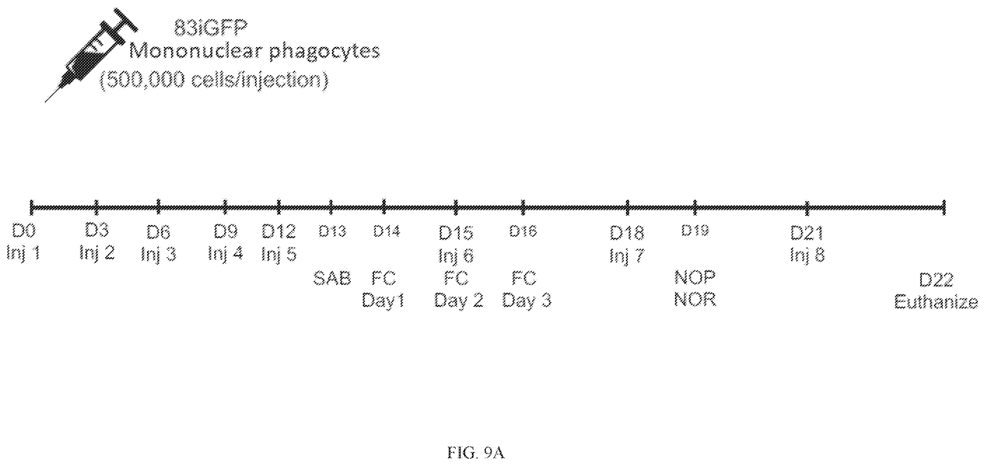

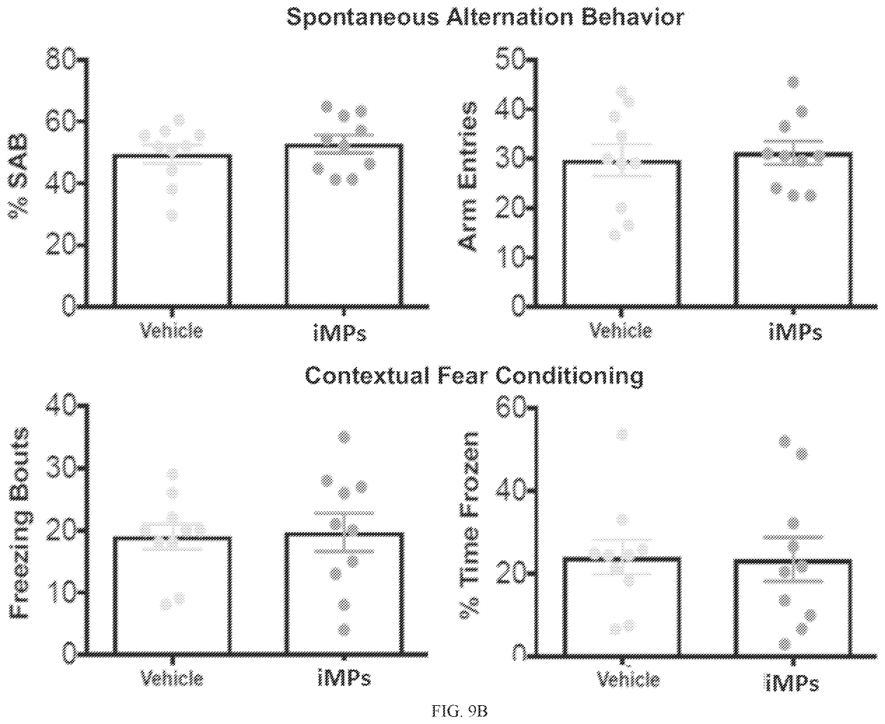

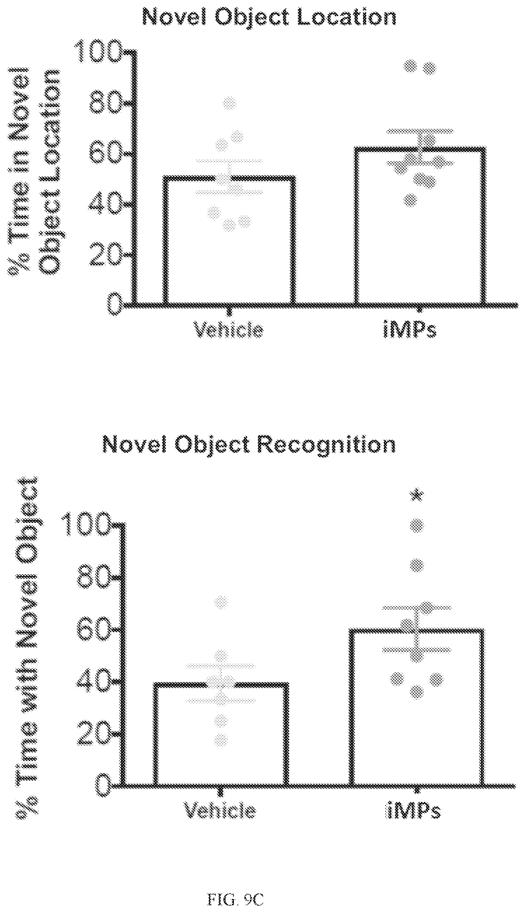

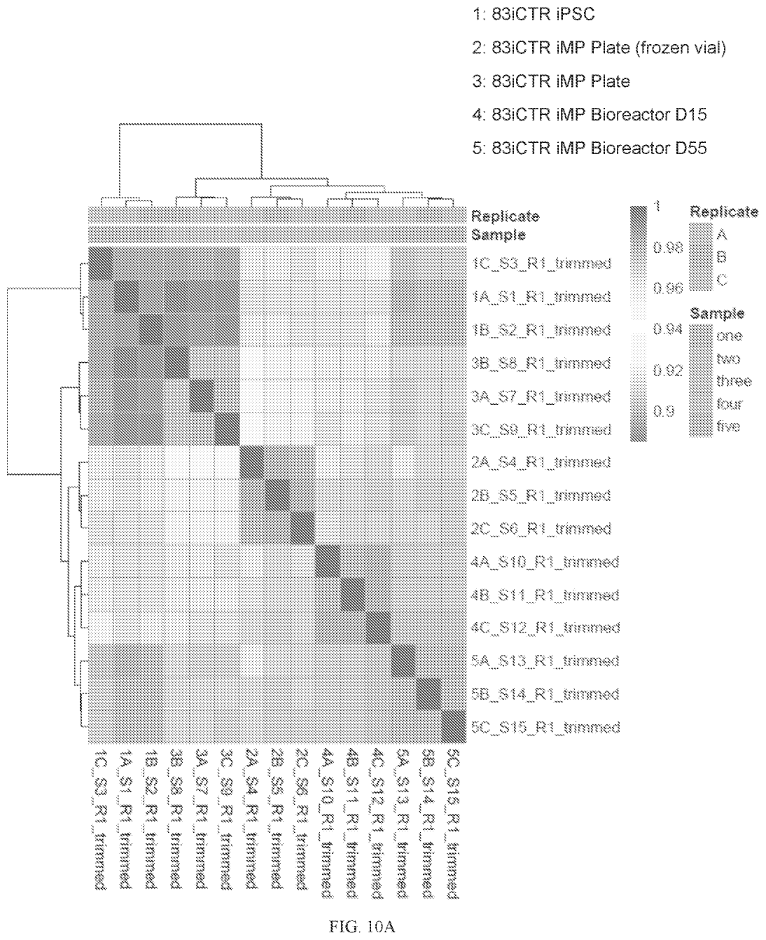

Additionally, we have begun testing the potential of iMPs in 5xFAD mice, a mouse model of Alzheimer's disease, (FIG. 9A) and found improvements in the novel object recognition study (FIG. 9C), even at 3 months of age when these mice were first developing pathology. We will also study 5xFAD mice at 8 months of age with significant pathology, and we expect that iMP treatment will more significantly affect behavioral and neural health/inflammation outcomes in these older 5xFAD mice.

These studies demonstrate the potential benefits of iMPs in improving cognition and neural health in aging and in neurodegeneration. iPSC-derived mononuclear phagocytes can mimic the effects of young plasma and bone marrow transplant and be used in subjects to restore cognitive function, as therapeutics in aging and Alzheimer's disease.

### Example 2

iMP is differentiated from iPSC using the following differentiation protocol. Passage iPSC using EZ Passage Tool at low density; aiming for 120 colonies in each well.

Chop only the center of the iPSC well using an EZ Passage Tool.

Aspirate media.

Using 3 mls of fresh media, blow the colonies off the plate. Avoid scraping!

Remove colony suspension and transfer to a new 15 ml conical tube. Dilute the colony suspension to be about 120 colonies/ml (eyeball). Note: one will need to dilute the colony suspension a lot (even up to adding 10 mls of media to the suspension).

Note: adjust concentration of colony suspension by cell line if needed (i.e. increase concentration for lines that have poor attachment and decrease for lines that have strong attachment).

Aspirate Matrigel from plates and replace with 1 ml of media/well.

Add 1 ml of colony suspension to each well, transfer to incubator and shake horizontally and vertically (z & x axis) 4 times each way.

Leave colonies to attach overnight.

### Colony Daily Maintenance

Clean out any differentiating cells and colonies that meet any of the following conditions:
Much smaller or larger than the majority of colonies
Has any differentiating cells next to it
Is too close to other colonies (i.e. would grow into each other over time)
The well has over 15 colonies (ideal well will have 10-12 colonies)
Any oddly shaped colonies (i.e. crescent moon shape/not close to circular at all)

It is important to have an even distribution of colonies that are even in diameter.

In bright field culture images (5X) representative of each differentiation stage, one should see similar colony morphology if differentiation is done properly. Also, note that as the differentiation progresses, the colonies become very “messy”, which is how they should be.

### Differentiation Start

Start stage 1 media (mTeSR*+80 ng/mL BMP4) when all colonies are minimum 0.7 mm in diameter and majority are 1.0 mm in diameter (7 centimeters diameter on evos 4X). This is D0 (day 0). * Unlike Douvaras et al. which used mTeSR custom medium as described in Stem Cell Reports, vol.8, 1516-1524, 2017, we use standard mTeSR (e.g., STEMCELL Technology, catalog number 85850, comprising at least bFGF and TGF (3). Douvaras et al. described in Stem Cell Reports, vol.8, 1516-1524, 2017 (Supplemental) that his mTeSR Custom medium is mTeSR1 medium without Lithium Chloride, GABA, Pipecolic Acid, basic fibroblast growth factor (bFGF), and transforming growth factor β (TGFβ1) (Stem Cell Technologies).

### Stage 1:

Stage 1 initiation is D0.

Feed 1 ml/well stage 1 media every day until day 4. That is, aspirate the supernatant each day and add 1 mL fresh media every day until day 4.

Full media change at the end of stage 1 into Stage 2 media immediately prior to Stage 2.

### Stage 2:

On day 4, switch cells to stage 2 media (StemPro-34 SFM+25 ng/mL basic fibroblast growth factor (bFGF), 80 ng/mL vascular endothelial growth factor (VEGF), 100 ng/mL stem cell factor (SCF)), 2 mls/well.

Full media change at the end of stage 2 immediately prior to Stage 3.

### Stage 3:

On day 6, switch cells to stage 3 (StemPro-34 SFM+50 ng/mL SCF, 50 ng/mL IL-3, 5 ng/mL thrombopoietin (TPO), 50 ng/mL macrophage CSF (M-CSF), 50 ng/mL FLT3-ligand (FLT3L)) 2 mls/well.

Full media change on day 10.

Media change on D10, cells were fed again (supernatant aspirated and fed fresh) with stage 3 medium at 2 mls/well.

Full media change at the end of Stage 3 immediately prior to Stage 4.

Note: Cyst formation starting to occur (a good sign).

### Stage 4 (Collection Phase):

On D12 or D13 or D14, switch cells to stage 4 media (StemPro-34 SFM+50 ng/mL M-CSF, 25 ng/mL GM-CSF, 50 ng/mL FLT3L) 2 mls/well (fully aspirating the supernatant and then adding stage 4 media).

No more aspiration subsequently, as cysts are loosely attached to the well plate, and we collected the cells that were floating in suspension, fed cells biweekly (i.e. every Monday or Tuesday and Friday), 2 mls/well stage 4. NO ASPIRATION.

Note: Cells will be fed twice a week, where one feed is post-collection. The cysts were loosely attached to the plate, and the monocytes or mononuclear phagocytes budded off of the cysts and floated in suspension. So once per week, the media containing the floating cells was collected via serological pipette and spun down, leaving a cell pellet, which we could then used for administration. The feeds should be 3 or 4 days apart; and the floating cells were collected and spun down, then the old media was aspirated, and the cell pellet was resuspended in fresh media and fed back to the plate. At this point in the protocol, Douvaras et al. (Stem Cell Reports, vol. 8, pp:1516-1524 Jun. 6, 2017) performed weekly sorting to collect only CD14+/CX3CR1+ cells and then continued to further differentiate these cells to microglia by exposing them to GM-CSF and IL34 for 2 weeks. Conversely, we did not sort these cells and instead collect this more immature cell type for use in treating our animal models of aging and neurodegeneration. We did not culture the cells in Microglial Medium (RPMI-1640 with 2 mM GlutaMAX-I, 10 ng/mL GM-CSF, and 100 ng/mL IL-34).

### Example 3

### Scale-Up Process in Bioreactor to Produce Clinically Relevant Numbers of iMPs and Characterizations

Once stage 4 was reached (about D14 in Example 2), the cells have formed cysts that are loosely attached to the plate. The iMPs budded off of these cysts and then floated in suspension. The iMP cysts were lifted, e.g., using a cell scraper, and transferred to a stirred flask bioreactor (e.g., CORNING®) on a low speed magnetic stir plate (e.g., DURA-MAG™). Lifted cysts were allowed to adjust to suspension culture for 24 hours after which time the stir plate was turned on and set to 30 rotations/min. In the bioreactor, the cysts floated and iMPs continued to bud off them, and the resulting iMPs were collected for further analysis or procedures.

We also fine-tuned a starting density: monolayer seeding density to be about 100,000 cells/cm². We used a polydimethylsiloxane (PDMS) stamp to plate small “islands” of proteins (e.g., extracellular matrix proteins or matrigel) for cells to attach to, i.e., “seeding” in discrete/isolated islands so that the cysts were spaced apart during the differentiation process, rather than all over the surface of a culturing device.

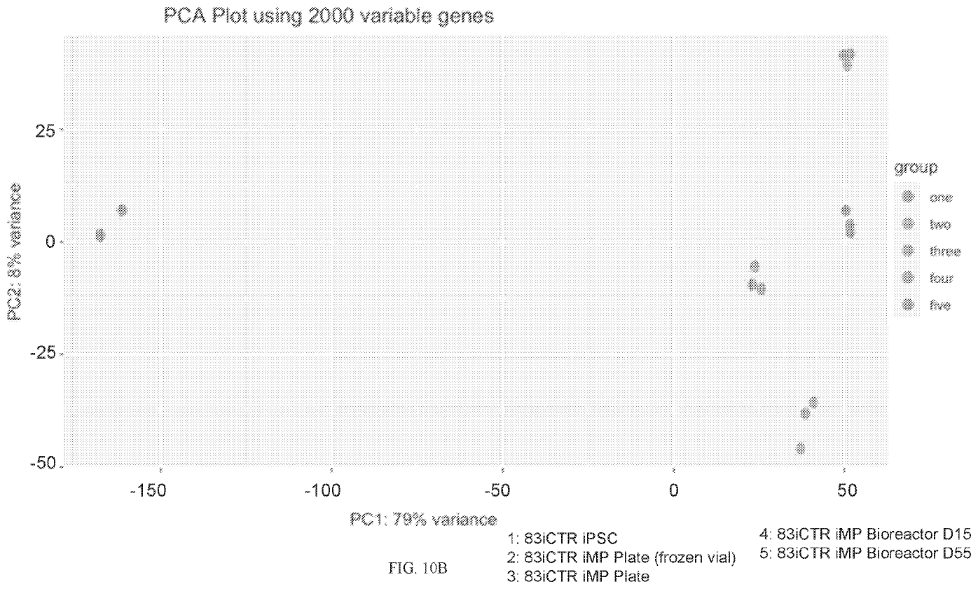

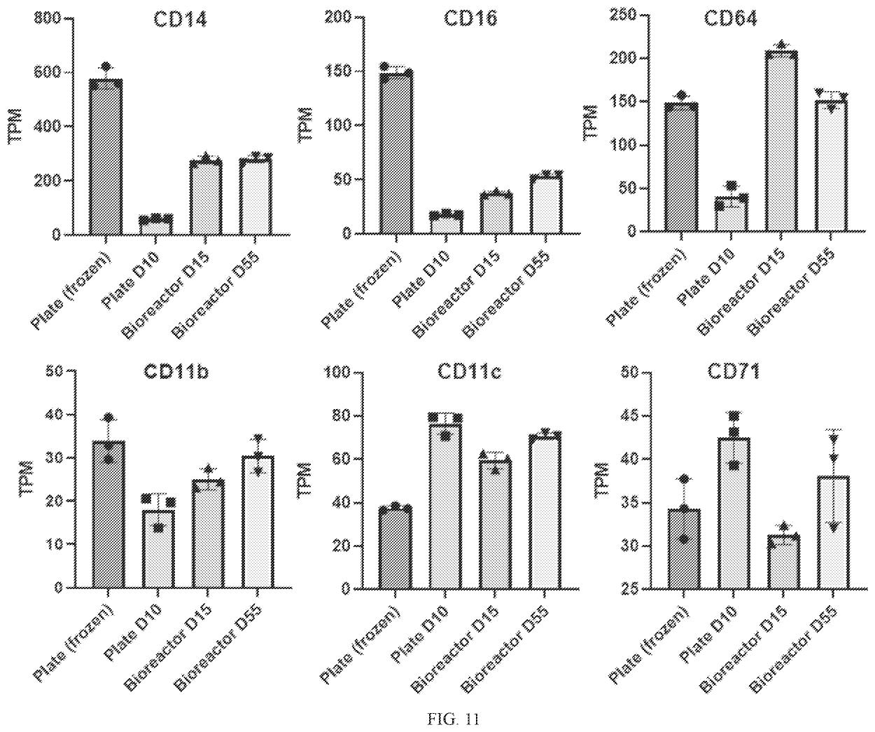

We also compared the gene expression, using RNA-sequencing techniques, of iMPs cultivated in the bioreactor for 15 days, iMPs cultivated in the bioreactor for 55 days, iMPs cultured in a well plate as shown in Example 2, and iMPs recovered from a frozen vial of cells cultured from a well plate as shown in Example 2, as well as iPSCs as a control. FIGS. 10A and 10B show that the iMPs in these four conditions were similar to each other in terms of hierarchical clustering analysis and principal component analysis. FIG. 11 shows the relative expression proportion of respective gene in each of the four groups.

We counted the total number of cells (including live and dead cells) collected at different days from the bioreactor cultivation, showing a consistently high viability of cells above 65% for at least 48 days (Table 1).

TABLE 1
The number of live cell vs. dead cells, and calculated
percentage of viability (viability = number of live
cells ÷ (number of live cells + number of dead
cells)) for each given date of collection from the bioreactor.

| Date | Live | Dead | Viability (%) |
|---|---|---|---|
| First date | 5,300,000 | 1,150,000 | 82.2 |
| First date + 7 days | 16,100,000 | 6,800,000 | 70.3 |
| First date + 12 days | 10,300,000 | 1,850,000 | 84.8 |
| First date + 18 days | 9,900,000 | 3,300,000 | 75 |
| First date + 28 days | 25,800,000 | 8,600,000 | 75 |
| First date + 38 days | 18,100,000 | 8,000,000 | 69.3 |
| First date + 48 days | 13,850,000 | 6,250,000 | 68.9 |

We have so far used either a 125 mL flask or a 500 mL flask but the process could scale past those volumes as needed.

In addition to RNA-Seq analysis, we also did flow cytometry, western blotting, and phagocytosis assay (bead uptake).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method for improving cognitive function in a subject, or treating a subject with a neurodegenerative disorder, comprising:
administering to the subject a therapeutically effective amount of a composition comprising mononuclear phagocytes generated from induced pluripotent stem cells (iPSCs), thereby improving the cognitive function in the subject or treating the subject with the neurodegenerative disorder,
wherein the mononuclear phagocytes are generated from the iPSCs in a process comprising culturing the iPSCs in a cell culture medium under conditions that induce myeloid differentiation, leading to the generation of mononuclear phagocytes, and
wherein the culturing does not include contacting the generated mononuclear phagocytes in a microglial differentiation medium comprising interleukin 34 (IL-34) or comprising IL-34 and granulocyte-macrophage colony-stimulating factor (GM-CSF), and the culturing does not include contacting the generated mononuclear phagocytes in dendritic cell differentiation medium comprising interleukin 4 (IL-4) or comprising IL-4 and GM-CSF.

2. The method of claim 1, wherein the mononuclear phagocytes comprises monocytes or express markers comprising CD11b, CD14, CD16, CD64, CD11c, CD71, or a combination thereof; and
optionally wherein after the administration, the mononuclear phagocytes produce macrophages in the subject, or optionally wherein the mononuclear phagocytes are further cultured in a cell culture medium under a condition that induces macrophage differentiation.

3. The method of claim 2, wherein the culturing comprises contacting the iPSCs with a first composition comprising bone morphogenetic protein (BMP-4), and wherein the culturing further comprises:
contacting the cell culture medium with a second composition after culturing of the iPSCs in the presence of the first composition, said second composition comprising one or more factors selected from the group consisting of bFGF, vascular endothelial growth factor (VEGF), stem cell factor (SCF), and a combination thereof;
contacting the cell culture medium with a third composition comprising one or more factors selected from the group consisting of SCF, interleukin 3 (IL-3), thrombopoietin (TPO), macrophage colony-stimulating factor (M-CSF), Fms-like tyrosine kinase 3 ligand (FLT3 ligand), and a combination thereof; and
contacting the cell culture medium with a fourth composition comprising one or more factors selected from the group consisting of M-CSF, GM-CSF, and FLT3 ligand, and a combination thereof.

4. (canceled)

5. (canceled)

6. (canceled)

7. The method of claim 3, comprising contacting the cell culture medium with the first composition for approximately 4 days, contacting the cell culture medium with the second composition for approximately 2 days, contacting the cell culture medium with the third composition for approximately 6 to 8 days, and/or contacting the cell culture medium with the fourth composition for approximately 3 to 90 days.

8. The method of claim 7, wherein the first composition comprising BMP-4 is in a mTeSR1 medium with one or more of bFGF, TGFβ, GABA, pipecolic acid, and lithium chloride; and the first composition is serum-free, and wherein the second composition, the third composition, and/or the fourth composition are independently in a serum-free hematopoietic cell medium, optionally being StemPro-34 serum-free medium.

9. (canceled)

10. The method of claim 1, wherein the process further comprises culturing in a bioreactor to obtain at least 1×10⁶, 5×10⁶, or 1×10⁷ of the mononuclear phagocytes.

11. The method of claim 1, wherein the iPSCs are derived from peripheral blood mononuclear cells (PBMCs) or fibroblasts obtained from the subject; and wherein the subject is a human of 50 years old or older with a neurodegenerative disorder selected from the group consisting of Alzheimer's disease, amyotrophic lateral sclerosis (ALS), Parkinson's disease, multiple sclerosis (MS), Rett syndrome, diffuse leukoenchephalopathy with spheroids, hereditary diffuse leukoenchephalopathy with axonal spheroids, frontotemporal lobar degeneration (FTLD), familial FTLD, schizophrenia, and autism spectrum disorders.

12. (canceled)

13. (canceled)

14. The method of claim 1, further comprising measuring in the subject after the administration one or more of: improved spatial working memory, improved short-term memory, increased synaptic transporter level, increased microglia branch length, of the subject, compared to a control subject or compared to respective level of the subject before the administration.

15. A method for generating mononuclear phagocytes comprising:
culturing induced pluripotent stem cells in a cell culture medium under conditions that induce myeloid differentiation, leading to the generation of mononuclear phagocytes, wherein the culturing comprises contacting the induced pluripotent stem cells with a first composition comprising bone morphogenetic protein (BMP-4),
contacting the cell culture medium with a second composition after culturing of the induced pluripotent stem cells in the presence of the first composition, said second composition comprising one or more factors selected from the group consisting of bFGF, vascular endothelial growth factor (VEGF), and stem cell factor (SCF), and a combination thereof,
contacting the cell culture medium with a third composition after culturing in the presence of the second composition, said third composition comprising one or more factors selected from the group consisting of SCF, interleukin 3 (IL-3), thrombopoietin (TPO), macrophage colony-stimulating factor (M-CSF), and Fms-like tyrosine kinase 3 ligand (FLT3 ligand), and a combination thereof, and
contacting the cell culture medium with a fourth composition after culturing in the presence of the third composition, said fourth composition comprising one or more factors selected from the group consisting of M-CSF, GM-CSF, and FLT3 ligand, and a combination thereof, thereby generating the mononuclear phagocytes,
wherein the culturing does not include contacting the generated mononuclear phagocytes in a microglial differentiation medium comprising interleukin 34 (IL-34) or comprising IL-34 and granulocyte-macrophage colony-stimulating factor (GM-CSF), and the culturing does not include contacting the generated mononuclear phagocytes in dendritic cell differentiation medium comprising interleukin 4 (IL-4) or comprising IL-4 and GM-CSF.

16. The method of claim 15, wherein the first, second, third, and fourth compositions are in serum-free medium; optionally the first composition is in a mTeSR1 medium and the second, third, and fourth compositions are in a StemPro-34 medium.

17. The method of claim 15, comprising contacting the cell culture medium with the first composition for approximately 4 days, contacting the cell culture medium with the second composition for approximately 2 days, contacting the cell culture medium with the third composition for approximately 6 to 8 days, and/or contacting the cell culture medium with the fourth composition for approximately 3 to 90 days.

18. The method of claim 15, wherein the culturing comprises culturing in a bioreactor to obtain a population of at least 1×10⁶, 5×10⁶, or 1×10⁷ of the mononuclear phagocytes; optionally the bioreactor being a stirrer tank bioreactor.

19. Mononuclear phagocytes generated using a method comprising:
(1) culturing pluripotent stem cells in an adherent culture in a first cell culture medium comprising bone morphogenetic protein 4 (BMP-4);
(2) culturing the cells obtained by step (1) in an adherent culture in a second cell culture medium comprising basic fibroblast growth factor (bFGF), vascular endothelial growth factor (VEGF), and stem cell factor (SCF);
(3) culturing the cells obtained by step (2) in an adherent culture in a third cell culture medium comprising SCF, interleukin 3 (IL-3), thrombopoietin, macrophage colony-stimulating factor (M-CSF), and FLT3 ligand; and
(4) culturing the cells obtained by step (3) in a suspension culture in a fourth cell culture medium comprising M-CSF, granulocyte-macrophage colony-stimulating factor (GM-CSF), and FLT3 ligand, wherein macrophages and/or monocytes are generated;
wherein the method does not include contacting the generated mononuclear phagocytes in a microglial differentiation medium comprising interleukin 34 (IL-34) or comprising IL-34 and GM-CSF, and the culturing does not include contacting the generated mononuclear phagocytes in dendritic cell differentiation medium comprising interleukin 4 (IL-4) or comprising IL-4 and GM-CSF; and
wherein the mononuclear phagocytes are positive for CD11b, CD14, CD16, CD64, CD11c, CD71, or a combination thereof.

20. The mononuclear phagocytes of claim 19, generated by the method wherein the pluripotent stem cells are human induced pluripotent stem cells.

21. The mononuclear phagocytes of claim 18, generated by the method wherein each of the first, second, third, and fourth cell culture media do not comprise serum;
optionally the first composition is in a mTeSR1 medium with or one or more, or all of basic fibroblast growth factor (bFGF), transforming growth factor β (TGFβ), aminobutyric acid (GABA), pipecolic acid, and lithium chloride, and optionally the second, third, and fourth compositions are in a StemPro-34 medium.

22. (canceled)

23. (canceled)

24. A method for treating a subject having a disease or disorder associated with a defect in or deficiency of macrophages, comprising administering to the subject a therapeutically effective amount of a composition comprising mononuclear phagocytes of claim 19, wherein the mononuclear phagocytes produce macrophages after the administration to the subject.

25. A method for generating mononuclear phagocytes and treating a subject with a neurodegenerative disorder or a subject in need of cognitive function improvement, comprising:
culturing induced pluripotent stem cells in a cell culture medium under conditions that induce myeloid differentiation, leading to the generation of mononuclear phagocytes, wherein the culturing comprises contacting the induced pluripotent stem cells with a first composition comprising bone morphogenetic protein (BMP-4),
contacting the cell culture medium with a second composition after culturing of the induced pluripotent stem cells in the presence of the first composition, said second composition comprising one or more factors selected from the group consisting of bFGF, vascular endothelial growth factor (VEGF), and stem cell factor (SCF), and a combination thereof,
contacting the cell culture medium with a third composition after culturing in the presence of the second composition, said third composition comprising one or more factors selected from the group consisting of SCF, interleukin 3 (IL-3), thrombopoietin (TPO), macrophage colony-stimulating factor (M-CSF), and Fms-like tyrosine kinase 3 ligand (FLT3 ligand), and a combination thereof, and
contacting the cell culture medium with a fourth composition after culturing in the presence of the third composition, said fourth composition comprising one or more factors selected from the group consisting of M-CSF, GM-CSF, and FLT3 ligand, and a combination thereof, thereby generating the mononuclear phagocytes,
wherein the culturing does not include contacting the generated mononuclear phagocytes in a microglial differentiation medium comprising interleukin 34 (IL-34) or comprising IL-34 and granulocyte-macrophage colony-stimulating factor (GM-CSF), and the culturing does not include contacting the generated mononuclear phagocytes in dendritic cell differentiation medium comprising interleukin 4 (IL-4) or comprising IL-4 and GM-CSF; and
administering to the subject a therapeutically effective amount of a composition comprising the generated mononuclear phagocytes, thereby treating the subject with the neurodegenerative disorder or improving the cognitive function in the subject.

26. The method of claim 25, wherein the culturing does not include contacting the generated mononuclear phagocytes in macrophage differentiation medium comprising M-CSF and one or both of interferon gamma (IFN-γ) and IL-4.

27. The method of claim 25, wherein the culturing further includes contacting the generated mononuclear phagocytes in macrophage differentiation medium, wherein the macrophage differentiation medium comprises M-CSF and IL-4 or comprises the M-CSF and interferon gamma (IFN-γ).

28. (canceled)

29. (canceled)

30. The method of claim 25, wherein the culturing comprises culturing in a bioreactor to generate a population of at least 1×10⁶, 5×10⁶, or 1×10⁷ of the mononuclear phagocytes, so as to administer the population of the generated mononuclear phagocytes to the subject.
